(19) United States
(12) Patent Application Publication
(10) Pub. No.: US 2026/0097008 A1
(43) Pub. Date: Apr. 9, 2026

(54) ACETYL LEUCINE FOR USE IN TREATING NIEMANN-PICK DISEASE TYPE C

(71) Applicant: IntraBio Inc., Austin, TX (US)

(72) Inventor: Michael STRUPP, Munich (DE)

(21) Appl. No.: 19/352,209

(22) Filed: Oct. 7, 2025

Related U.S. Application Data
(60) Provisional application No. 63/704,296, filed on Oct. 7, 2024.
(60) Provisional application No. 63/811,423, filed on May 23, 2025.

(51) Int. Cl. A61K 31/198; A61K 31/445; A61P 25/28

(52) U.S. Cl. CPC A61K 31/198; A61K 31/445; A61P 25/28

(57) ABSTRACT

The present disclosure provides methods of treating NPC, or a neurological symptom thereof, in a subject by administering a therapeutically effective amount of N-acetyl leucine, wherein the subject has one or more NPC1 mutations.

### BACKGROUND

### Field

The present disclosure provides methods of treating or preventing Niemann-Pick disease type C, or a neurological symptom thereof, in a subject by administering a therapeutically effective amount of N-acetyl leucine, or a pharmaceutically acceptable salt thereof, wherein the subject has one or more NPC1 gene mutations.

### Background

Niemann-Pick disease type C (NPC) disease is a rare, progressive, debilitating, and prematurely fatal autosomal recessive lysosomal storage disorder (LSD), with an incidence of one case per 100,000 persons. The disease manifests with systemic, psychiatric, and neurologic symptoms, and many aspects of neurologic function are impaired.

NPC is a genetically and clinically heterogeneous disease. A broad phenotypic spectrum has been described, with differences in the age of onset of symptoms, rate of progression, disease severity, organs affected, effects on the central nervous system, and response to pharmacological treatments. See, e.g., Las Heras et al., npj Genomic Medicine (2023) 8:21; https://doi.org/10.1038/s41525-023-00365-w. At present, treatment of NPC is limited to slowing the progression of neurologic symptoms with miglustat, a drug used in substrate reduction therapy for glycosphingolipid lysosomal storage disorders. Miglustat has been approved in several countries but not in the United States.

There exists a need in the art for safe and efficacious drugs to treat NPC subjects having specific NPC1 gene mutations and/or clinical phenotypes.

### BRIEF SUMMARY

In one aspect, the present disclosure provides methods of treating or preventing Niemann-Pick disease type C or “NPC,” or a neurological symptom thereof, comprising administering a therapeutically effective amount of N-acetyl leucine, e.g., N-acetyl-DL-leucine or N-acetyl-L-leucine, to the subject, wherein the subject has one or more NPC1 gene mutations.

In another aspect, the present disclosure provides N-acetyl leucine, or a pharmaceutical composition thereof, for use in treating or preventing NPC, or a neurological symptom thereof, in a subject in need thereof, wherein the subject has one or more NPC1 gene mutations.

In another aspect, the present disclosure provides the use of N-acetyl leucine for the manufacture of a medicament for treating or preventing NPC, or a neurological symptom thereof, in a subject in need thereof, wherein the subject has one or more NPC1 gene mutations.

In another aspect, the present disclosure provides a method of providing neuroprotection in a subject in need thereof, the method comprising administering a therapeutically effective amount of N-acetyl leucine, e.g., N acetyl-DL-leucine or N-acetyl-L-leucine, to the subject, wherein the subject has NPC, or a neurological symptom thereof, and/or the subject has one or more NPC1 gene mutations.

It is to be understood that both the foregoing summary and the following detailed description are exemplary and explanatory only, and are not restrictive of the invention as claimed.

### BRIEF DESCRIPTION OF THE DRAWINGS

FIG. 1 is an illustration showing the clinical study scheme described in EXAMPLE 1.

FIG. 2A is a spaghetti plot showing the SARA Total Score versus Visit per Treatment Sequence. The straight lines represent NALL treatment; the dashed lines represent placebo treatment. 2=Visit 2; 4=Visit 4; 6=Visit 6.

FIG. 2B is a spaghetti plot showing the mSARA Total Score versus Visit per Treatment Sequence. The straight lines represent NALL treatment; the dashed lines represent placebo treatment. 2=Visit 2; 4=Visit 4; 6=Visit 6.

FIG. 3 is a forest plot showing the SARA total score at Visit 4 vs Visit 6 with 95% confidence intervals. The dots represent mean of the differences between NALL and Placebo, the horizontal lines represent the 95% confidence intervals. * Least squares mean difference from primary analysis. **Observed mean of treatment differences.

FIG. 4A is a bar graph showing the results of the EQ-5D-5L (patients>18 years old) questionnaire with respect to mobility.

FIG. 4B is a bar graph showing the results of the EQ-5D-5L (patients>18 years old) questionnaire with respect to self-care.

FIG. 4C is a bar graph showing the results of the EQ-5D-5L (patients>18 years old) questionnaire with respect to usual activities.

FIG. 4D is a bar graph showing the results of the EQ-5D-5L (patients>18 years old) questionnaire with respect to pain/discomfort.

FIG. 4E is a bar graph showing the results of the EQ-5D-5L (patients>18 years old) questionnaire with respect to anxiety/depression.

FIG. 5A is a bar graph showing the results of the EQ-5D-Y (patients<18 years old) questionnaire with respect to mobility.

FIG. 5B is a bar graph showing the results of the EQ-5D-Y (patients<18 years old) questionnaire with respect to self-care.

FIG. 5C is a bar graph showing the results of the EQ-5D-Y (patients<18 years old) questionnaire with respect to usual activities.

FIG. 5D is a bar graph showing the results of the EQ-5D-Y (patients<18 years old) questionnaire with respect to pain/discomfort.

FIG. 5E is a bar graph showing the results of the EQ-5D-Y (patients<18 years old) questionnaire with respect to anxiety/depression.

FIG. 6 is a line graph showing the mean (+/−standard error) of the fSARA total score by time and treatment sequence.

FIG. 7 is an illustration showing the clinical study scheme described in EXAMPLE 3.

FIG. 8 is a bar graph showing the 5-Domain NPC-CSS Total Score versus Published Historical Cohorts & Clinical Trial Cohorts. Percentage calculated based on the annualized progression rate of 1.5 points (representing a −100% rate of annual progression); a negative value reflects disease worsening/progression; 0 reflects no change (disease stabilization); and a positive value reflects disease improvement.

### DETAILED DESCRIPTION

A “subject,” as used herein, may be a vertebrate, mammal or domestic animal. Hence, compositions according to the disclosure may be used to treat any mammal, for example livestock, e.g. a horse, cow, sheep or pig, pets, e.g. a cat, dog, rabbit or guinea pig, a laboratory animal, e.g. a mouse or rat, or may be used in other veterinary applications. In one embodiment, the subject is a human being. “Subject” and “patient” are used interchangeably.

As used herein, the singular forms “a,” “an,” and “the” include plural reference.

As used herein, the term “about” should be generally understood to encompass ±10% of a specified amount, frequency or value. Numerical quantities given herein are approximate unless stated otherwise, meaning that term “about” can be inferred when not expressly stated.

The terms “administer,” “administration,” or “administering” as used herein refer to (1) providing, giving, dosing and/or prescribing by either a health practitioner or his authorized agent or under his direction, N-acetyl leucine; and (2) putting into, taking or consuming by the patient or person himself or herself, N-acetyl leucine.

The term “N-acetyl leucine” refers to N-acetyl-DL-leucine, N-acetyl-D-leucine, and/or N-acetyl-L-leucine. Any reference to N-acetyl leucine includes pharmaceutically acceptable salts of the same, even if not expressly stated. In some embodiments, the N-acetyl leucine is N-acetyl-L-leucine or “NALL.” N-acetyl-L-leucine may also be referred to as “IB1001” or “AQNEURSA.”

The term “neuroprotection” as used herein, refers to prevention, a slowing in, and/or a reversed progression of neurodegeneration, including, but not limited to, progressive loss of neuronal structure, progressive loss of neuronal function, and/or progressive neuronal death. Providing neuroprotection may result in delaying onset of NPC, or one or more symptoms of NPC that would otherwise be expected to manifest according to typical disease progression, reducing the severity of NPC or reducing the severity of or eliminating one or more existing symptoms associated with NPC, delaying progression of NPC, or one or more symptoms of NPC over time as compared to typical disease progression, and/or reversing progression of NPC, or one or more symptoms of NPC over time. The time over which neuroprotection is provided may coincide with the duration of treatment with NALL. The treatment may provide neuroprotection over a duration of, for example, about one month or more, about two months or more, about three months or more, about four months or more, about five months or more or about six months or more, about 1 year or more, about 2 years or more, about 3 years or more, about 4 years or more, about 5 years or more, or about 10 years or more. The treatment may provide neuroprotection over the lifetime of the patient.

A “pharmaceutically acceptable salt” as referred to herein, is any salt preparation that is appropriate for use in a pharmaceutical application. Pharmaceutically acceptable salts include, but are not limited to, amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

N-acetyl leucine may be formulated and administered to a subject in accordance with known teachings in the art. For example, N-acetyl leucine may be formulated as a pharmaceutical composition. The pharmaceutical composition may comprise N-acetyl-DL-leucine and a pharmaceutically acceptable carrier, N-acetyl-L-leucine and a pharmaceutically acceptable carrier, or N-acetyl-D-leucine and a pharmaceutically acceptable carrier.

The pharmaceutical composition may take any of a number of different forms depending, in particular, on the manner in which it is to be used. Thus, for example, it may be in the form of a powder, tablet, capsule, liquid, ointment, cream, gel, hydrogel, aerosol, spray, micellar solution, transdermal patch, liposome suspension or any other suitable form that may be administered to a person or animal in need of treatment.

A “pharmaceutically acceptable carrier” as referred to herein, is any known compound or combination of known compounds, e.g., excipients, carriers, etc., that are known to those skilled in the art to be useful in formulating pharmaceutical compositions. It will be appreciated that the carrier of the pharmaceutical composition should be one which is tolerated by the subject to whom it is given.

In one embodiment, the pharmaceutically acceptable carrier may be a solid, and the composition may be in the form of a powder or tablet. A solid pharmaceutically acceptable carrier may include, but is not limited to, one or more substances which may also act as flavouring agents, buffers, lubricants, stabilisers, solubilisers, suspending agents, wetting agents, emulsifiers, dyes, fillers, glidants, compression aids, inert binders, sweeteners, preservatives, dyes, coatings, or tablet-disintegrating agents. The carrier may also be an encapsulating material. In powders, the carrier may be a finely divided solid that is in admixture with the finely divided active agents according to the disclosure. In tablets, the active agent may be mixed with a carrier having the necessary compression properties in suitable proportions and compacted in the shape and size desired. The powders and tablets may, for example, contain up to 99% of the active agents. Suitable solid carriers include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutically acceptable carrier may be a gel and the composition may be in the form of a cream or the like.

The carrier may include, but is not limited to, one or more excipients or diluents. Examples of such excipients are gelatin, gum arabicum, lactose, microcrystalline cellulose, starch, sodium starch glycolate, calcium hydrogen phosphate, magnesium stearate, talcum, colloidal silicon dioxide, and the like.

In another embodiment, the pharmaceutically acceptable carrier may be a liquid. In one embodiment, the pharmaceutical composition is in the form of a solution. Liquid carriers are used in preparing solutions, suspensions, emulsions, syrups, elixirs and pressurized compositions. N-acetyl leucine may be dissolved or suspended in a pharmaceutically acceptable liquid carrier such as water, an organic solvent, a mixture of both or pharmaceutically acceptable oils or fats. The liquid carrier may contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid carriers for oral and parenteral administration include water (partially containing additives as above, e.g. cellulose derivatives, such as sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier may also be an oily ester such as ethyl oleate and isopropyl myristate. Sterile liquid carriers are useful in sterile liquid form compositions for parenteral administration. The liquid carrier for pressurized compositions may be a halogenated hydrocarbon or other pharmaceutically acceptable propellant.

Liquid pharmaceutical compositions, which are sterile solutions or suspensions, may be utilized by, for example, intramuscular, intrathecal, epidural, intraperitoneal, intravenous and subcutaneous injection. The active agent may be prepared as a sterile solid composition that may be dissolved or suspended at the time of administration using sterile water, saline, or other appropriate sterile injectable medium.

The compositions may be administered orally in the form of a sterile solution or suspension optionally containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compositions may also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Compositions may alternatively be administered by inhalation, e.g. intranasally. Compositions may also be formulated for topical use. For instance, creams or ointments may be applied to the skin.

N-Acetyl leucine may be incorporated within a slow- or delayed-release device. Such devices may, for example, be inserted on or under the skin, and the medicament may be released over weeks or even months. Such devices may be advantageous when long-term treatment with N-acetyl leucine according to the present disclosure is required and which may require frequent administration, e.g. at least daily administration.

In one embodiment, the pharmaceutical composition is a solid oral dosage form, such as a tablet. In tablets, the active agent may be mixed with a vehicle, such as a pharmaceutically acceptable carrier, having the necessary compression properties in suitable proportions and compacted in the shape and size desired. The tablets may contain up to 99% by weight of the N-acetyl leucine.

Pharmaceutical compositions in solid oral dosage form, such as tablets, may be prepared by any method known in the art of pharmacy. Pharmaceutical compositions are usually prepared by mixing the active agent with conventional pharmaceutically acceptable carriers.

A tablet may be formulated as is known in the art. Tanganil®, for example, includes wheat starch, pregelatinised maize (corn) starch, calcium carbonate and magnesium stearate as excipients. The same, or similar, excipients, for example, may be employed with the present disclosure.

The composition of each 700 mg Tanganil® tablet is as follows: 500 mg acetyl-DL-leucine, 88 mg wheat starch, 88 mg pregelatinised maize (corn) starch, 13 mg calcium carbonate and 11 mg magnesium stearate. The same tablets, for example, may be employed in the methods of the present disclosure.

In one embodiment, N-acetyl-L-leucine is formulated as granules for oral suspension in sachet. The granules for oral suspension may also comprise isomalt and/or hypromellose. Each sachet with 1000 mg of N-acetyl-L-leucine may be formulated, for example, to be suspended in 40 mL water.

As discussed above, N-acetyl leucine may be formulated and administered as a pharmaceutical composition taking any number of different forms. For example, N-acetyl leucine may be formulated as a pharmaceutical composition to facilitate its delivery across the blood-brain barrier. As a further example, N-acetyl leucine may be formulated as a pharmaceutical composition for bypassing the blood-brain barrier. Formulations that facilitate delivery across the blood-brain barrier or that are suitable for administration in a manner that bypasses the blood-brain barrier may be used to prepare and administer N-acetyl leucine.

In one embodiment, the pharmaceutical composition, e.g., a pharmaceutical composition comprising N-acetyl-L-leucine, or salt thereof, is formulated for nanodelivery, e.g., colloidal drug-carrier systems. Suitable examples include but are not limited to liposomes, nanoparticles (e.g., polymeric, lipid and inorganic nanoparticles), nanogels, dendrimers, micelles, nanoemulsions, polymersomes, exosomes, and quantum dots. See, e.g., Patel et al., “Crossing the Blood-Brain Barrier: Recent Advances in Drug Delivery to the Brain,” CNS Drugs 31:109-133 (2017); Kabanov et al., “New Technologies for Drug Delivery across the Blood Brain Barrier,” Curr Pharm Des., 10 (12): 1355-1363 (2004); Cheng et al., “Highly Stabilized Curcumin Nanoparticles Tested in an In Vitro Blood-Brain Barrier Model and in Alzheimer's Disease Tg2576 Mice,” The AAPS Journal, vol. 15, no. 2, pp. 324-336 (2013); Lähde et al. “Production of L-Leucine Nanoparticles under Various Conditions Using an Aerosol Flow Reactor Method,” Journal of Nanomaterials, vol. 2008, article ID 680897 (2008).

In one embodiment, the pharmaceutical composition, e.g., a pharmaceutical composition comprising acetyl-L-leucine, or salt thereof, is formulated for direct delivery to the central nervous system (CNS), such as by injection or infusion. Formulations for and methods of direct delivery to the CNS are known in the art. See, e.g., U.S. Pat. No. 9,283,181. Examples of such administration include but are not limited to intranasal, intraventricular, intrathecal, intracranial, and delivery via nasal mucosal grafting.

In one embodiment, the pharmaceutical composition is formulated for (and administered by) intranasal delivery. See, e.g., Hanson et al., “Intranasal delivery bypasses the blood-brain barrier to target therapeutic agents to the central nervous system and treat neurodegenerative disease,” BMC Neurosci. 9 (Suppl 3): S5 (2008). In one embodiment, the pharmaceutical composition is formulated for (and administered by) delivery via a nasal mucosal graft. In one embodiment, the pharmaceutical composition is formulated for (and administered by) intracerebroventricular injection or infusion. In another embodiment, the pharmaceutical composition is formulated for (and administered by) intrathecal intracisternal injection or infusion. In one embodiment, the pharmaceutical composition is formulated for (and administered by) intrathecal lumbar injection or infusion.

Various techniques may be used including, without limitation, injection through a burrhole or cisternal or lumbar puncture or the like as known in the art. Various devices, whether internal (e.g., implanted) or external, may be used for delivery as known in the art, such as pumps, catheters, reservoirs, etc. In one embodiment, the administration interval is once every two weeks.

In one embodiment, the administration interval is once every month. In one embodiment, the administration interval is once every two months. In one embodiment, the administration interval is twice per month. In one embodiment, the administration interval is once every week. In one embodiment, the administration interval is twice or several times per week. In one embodiment, the administration interval is daily. In one embodiment, the administration is continuous, such as continuous infusion.

In one embodiment, the dose or amount equivalent of N-acetyl leucine may adjusted to account for either its direct delivery to the CNS or its delivery across the blood-brain barrier.

A “subject in need thereof” as used herein may be any subject who has NPC, or a neurological symptom thereof.

In one embodiment, the present disclosure provides N-acetyl-DL-leucine, or a pharmaceutical composition thereof, for treating NPC in a subject in need thereof, wherein the subject has one or more NPC1 gene mutations.

In another embodiment, the present disclosure provides N-acetyl-DL-leucine, or a pharmaceutical composition thereof, for treating a neurological symptom of NPC in a subject in need thereof, wherein the subject has one or more NPC1 gene mutations.

In another embodiment, the present disclosure provides N-acetyl-L-leucine for the treatment of neurological manifestations of NPC in an adult subject or pediatric subject weighing 15 kg or more, wherein the adult or pediatric subject has one or more NPC1 gene mutations.

In one embodiment, the present disclosure provides N-acetyl-L-leucine, or a pharmaceutical composition thereof, for treating NPC in a subject in need thereof, wherein the subject has one or more NPC1 gene mutations.

In another embodiment, the present disclosure provides N-acetyl-L-leucine, or a pharmaceutical composition thereof, for treating a neurological symptom of NPC in a subject in need thereof, wherein the subject has one or more NPC1 gene mutations.

In one embodiment, the present disclosure provides N-acetyl-DL-leucine, or a pharmaceutical composition thereof, for preventing NPC in a subject in need thereof, wherein the subject has one or more NPC1 gene mutations.

In another embodiment, the present disclosure provides N-acetyl-DL-leucine, or a pharmaceutical composition thereof, for preventing a neurological symptom of NPC in a subject in need thereof, wherein the subject has one or more NPC1 gene mutations.

In one embodiment, the present disclosure provides N-acetyl-L-leucine, or a pharmaceutical composition thereof, for preventing NPC in a subject in need thereof, wherein the subject has one or more NPC1 gene mutations.

In another embodiment, the present disclosure provides N-acetyl-L-leucine, or a pharmaceutical composition thereof, for preventing a neurological symptom of NPC in a subject in need thereof, wherein the subject has one or more NPC1 gene mutations.

Exemplary non-limiting NPC1 gene mutations include any one or more of the mutations listed in Table 1 and/or Table 6.

TABLE 1
NPCI Gene Mutations

c.3019C > G p.(Pro1007Ala)
c.1654 + 5G > A, p.(?)
c.275A > G (p.Gln92Arg)
c.1918G > A (p.Gly640Arg)
c.[1918G > A](p.[Gly640Arg](+)[3451G > A]p.Ala1151Thr)
c.2861C > T, p.(Ser954Leu)
c.1574A > T p.(asp525Val)
c.306T > G p.Tyr102*
c.180G > T p.Gln60His
homozygous c.1918G > A (p.Gly640Arg)
c.3451G > A (p.Ala1151Thr)
c.2474 A > G p.(Tyr825Cys)
c.2780C > T (p.Ala927Val)
c.1295delG (p.Gly432Aspfs*17)
homozygous T3128 −> C (Ile1061 −> Thr)
p.I1061T
p.D948N
Homozygous c.3246-5_3246-7del
c.2819C < T(p.Ser940Leu)
c.2657G > T
c.2932C > T
c.2692G > A
c.3773delCC
homozygous c.3122A > G (p.Tyr1041Cys)
homozygous p.Val1165Met
homozygous c.2662C > T(p.P888S)
c.478T > C (p.C160R)
c.665A > G (p.N222S)
c.3106A > G (p.T1036A)
c.2861C > T p.(Ser954Leu) in exon 19
c.3027del p.(Lys1010Serfs*30) in exon 20
c.808 delG (+) c.2861 C > T
c.[2861C > T]
c.[3182T > C]
c.[2096T > A]
c.[2861C > T]
c.[2780 C > T]
c.[ 2780 C > T]
c. [2039dupT]
heterozygous c1947 + 5 G > C, c. 3107 C > T
heterozygous c1554-1009 G > A, c3182T > C p(Ile1081Thr)
homozygous c.1408G > C p.(Ala470Pro)
homozygous c.2509A > G p.(Ile837Val)
compound heterozygous c.3182T > C p.(Ile1061Thr)
compound heterozygous c.3611_3614del p.(Leu1204Glnfs*37)
compound heterozygous c.2713C > A p.(Gln905Lys)
compound heterozygous c.3182T > C p.(Ile1061Thr)
c.3493G > A (p.Val1165Met) in exon 23
p.Ser901Tyr + p.Asn222Ser
c.1554-1009G > A
compound heterozygous c.3182T.C p.(IIe1061Thr)
compound heterozygous c.3019C.G p.(Pro1007Ala)
c.2660C > T (p.Pro887Leu)
c.3019C > G (p.Pro1007Ala)
homozygous c.3394G > C p.A1132P
heterozygous chr18: 21115645 C > G p.Glu1089Gln
heterozygous chr. 18: 21118528 G > C p.Pro1007Ala
heterozygous c.3019C > G p.P1007A
heterozygous c.3104C > T p.A1035V
heterozygous c.2762A > G p.Q921P
heterozygous c.3106A > G p.T1036A
heterozygous c.3182T.C p.I1061T
heterozygous c.3104C > T p.A1035V
homozygous c.2728G > A pGly910Ser
heterozygous c.1211G > A p.R404Q
heterozygous c.3493G > A p.V1165M
heterozygous c.1114C > T p.R372W
heterozygous c.3322dupG p.A1108Gfs*13
[c.3182T > C, (p.I1061T)]
[c.2893C > T, (p.Q965*)]
[c.2849T > C, (p.V950G)]
homozygous c.3019C > G, (p.P1007A)
[c.2090T > C, (p.V697A)]
[c.3246-25A > G]
[c.1926G > C, (p.M641I)]
heterozygous p.R615C
heterozygous p.G1015V
homozygous c.1654 + 6T > A
heterozygous p.Arg518Trp
heterozygous p.Asp1156Ser
heterozygous p.Asp1097Asn
heterozygous p.Tyr1088Cys

In some embodiments, the NPC1 gene mutation is not c.808 delG (+) c.2861 C>T, c.[2861C>T], or c.[3182T>C].

In another embodiment, the present disclosure provides N-acetyl-L-leucine, or a pharmaceutical composition thereof, for treating a neurological symptom of NPC in a subject in need thereof, wherein the subject has one or more NPC1 gene mutations.

In another embodiment, the present disclosure provides N-acetyl-L-leucine, or a pharmaceutical composition thereof, for providing neuroprotection in a subject in need thereof, wherein the subject has NPC.

In another embodiment, the present disclosure provides N-acetyl-L-leucine, or a pharmaceutical composition thereof, for providing neuroprotection in a subject in need thereof, wherein the subject has one or more NPC1 gene mutations.

Exemplary non-limiting neurological symptoms of NPC include any one or more of the symptoms listed in Table 2 and/or Table 6.

TABLE 2

cognitive impairment
cognitive decline
cerebellar ataxia
clumsiness
attention-deficit/hyperactivity disorder (ADHD)
dystonia
supranuclear gaze palsy
ataxia
dysarthrophonia
epilepsy
cataplexy
vertical gaze palsy
dyspraxia
tremor
gelastic cataplexy
vertical supranuclear gaze palsy
daytime somnolence
dysarthria
dysphagia
insomnia
supraventricular gaze palsy
hepato-splenomegaly
muscular hypotonia
executive impairment
memory impairment

In some embodiments, the subject has been diagnosed with NPC by genetic testing prior to administration of N-acetyl leucine. In some embodiments, genetic testing reveals the subject has one or more NPC1 gene mutations, e.g., any one or more of the NPC1 mutations listed in Table 1 or Table 6.

In some embodiments, the subject is less than two years old.

In some embodiments, the subject is two years old to less than six years old.

In some embodiments, the subject is six years old to less than 15 years old.

In some embodiments, the subject is 15 years old or more.

In some embodiments, the subject weighs 5 kg or more.

In some embodiments, the subject weights 5 kg to <15 kg.

In some embodiments, the subject weighs 15 kg or more.

In some embodiments, the subject weighs 15 kg to <25 kg.

In some embodiments, the subject weighs 25 kg or more.

In some embodiments, the subject weighs 25 kg to <35 kg.

In some embodiments, the subject weighs 35 kg or more.

In some embodiments, administering N-acetyl-DL-leucine or N-acetyl-L-leucine to the subject results in an improvement over baseline in at least one clinical assessment of neurological signs and symptoms of NPC, e.g., at least one clinical assessment used to measure the severity of neurological signs and symptoms of NPC.

In some embodiments, the clinical assessment comprises one or more the Scale for the Assessment and Rating of Ataxia (SARA), the modified SARA (mSARA), the functional SARA (fSARA), see EXAMPLE 2, the modified Disability Rating Scale (mDRS), the timed 8-Meter Walk Test (8MWT), the timed 9-Hole Peg Test (9HPT) Niemann-Pick type C Clinical Severity Scale (NPC-CSS), 5-Domain NPC-CSS, and/or the Spinocerebellar Ataxia Functional Index (SCAFI).

In some embodiments, the clinical assessment is the Scale for the Assessment and Rating of Ataxia (SARA), the modified SARA (mSARA), the functional SARA (fSARA), the modified Disability Rating Scale (mDRS), the timed 8-Meter Walk Test (8MWT), the timed 9-Hole Peg Test (9HPT) Niemann-Pick type C Clinical Severity Scale (NPC-CSS), 5-Domain NPC-CSS, and/or the Spinocerebellar Ataxia Functional Index (SCAFI).

In some embodiments, the clinical assessment is the SARA.

In some embodiments, the SARA comprises a change from baseline in the SARA total score of at least −0.2.

In some embodiments, the SARA comprises a change from baseline in the SARA total score of at least −0.4.

In some embodiments, the SARA comprises a change from baseline in the SARA total score is at least −0.6.

In some embodiments, the SARA comprises a change from baseline in the SARA total score of at least −0.8.

In some embodiments, the SARA comprises a change from baseline in the SARA total score of at least −1.0.

In some embodiments, the SARA comprises a change from baseline in the SARA total score of at least −1.2.

In some embodiments, the SARA comprises a change from baseline in the SARA total score of at least −1.4.

In some embodiments, the SARA comprises a change from baseline in the SARA total score of at least −1.6

In some embodiments, the SARA comprises a change from baseline in the SARA total score of at least −1.8.

In some embodiments, the SARA comprises a change from baseline in the SARA total score of at least −2.0.

In some embodiments, the SARA comprises a change from baseline in the SARA total score of at least −2.2.

In some embodiments, the SARA comprises a change from baseline in the SARA total score of at least −2.4.

In some embodiments, the SARA comprises a change from baseline in the SARA total score is at least −2.6.

In some embodiments, the SARA comprises a change from baseline in the SARA total score of at least −2.8.

In some embodiments, the SARA comprises a change from baseline in the SARA total score of at least −3.0.

In some embodiments, the SARA comprises a change from baseline in the SARA total score of at least −3.2.

In some embodiments, the SARA comprises a change from baseline in the SARA total score of at least −3.4.

In some embodiments, the SARA comprises a change from baseline in the SARA total score of at least −3.6.

In some embodiments, the SARA comprises a change from baseline in the SARA total score of at least −3.8.

In some embodiments, the SARA comprises a change from baseline in the SARA total score of at least −4.0.

In some embodiments, the clinical assessment is the mSARA.

In some embodiments, the clinical assessment is the fSARA.

In some embodiments, the fSARA comprises a change from baseline in the fSARA total score of at least −0.2.

In some embodiments, the fSARA comprises a change from baseline in the fSARA total score of at least −0.4.

In some embodiments, the fSARA comprises a change from baseline in the fSARA total score is at least −0.6.

In some embodiments, the fSARA comprises a change from baseline in the fSARA total score of at least −0.8.

In some embodiments, the fSARA comprises a change from baseline in the fSARA total score of at least −1.0.

In some embodiments, the fSARA comprises a change from baseline in the fSARA total score of at least −1.2.

In some embodiments, the fSARA comprises a change from baseline in the fSARA total score of at least −1.4.

In some embodiments, the fSARA comprises a change from baseline in the fSARA total score of at least −1.6

In some embodiments, the fSARA comprises a change from baseline in the fSARA total score of at least −1.8.

In some embodiments, the fSARA comprises a change from baseline in the fSARA total score of at least −2.0.

In some embodiments, the fSARA comprises a change from baseline in the fSARA total score of at least −2.2.

In some embodiments, the fSARA comprises a change from baseline in the fSARA total score of at least −2.4.

In some embodiments, the fSARA comprises a change from baseline in the fSARA total score is at least −2.6.

In some embodiments, the fSARA comprises a change from baseline in the fSARA total score of at least −2.8.

In some embodiments, the fSARA comprises a change from baseline in the fSARA total score of at least −3.0.

In some embodiments, the fSARA comprises a change from baseline in the fSARA total score of at least −3.2.

In some embodiments, the fSARA comprises a change from baseline in the fSARA total score of at least −3.4.

In some embodiments, the fSARA comprises a change from baseline in the fSARA total score of at least −3.6.

In some embodiments, the fSARA comprises a change from baseline in the fSARA total score of at least −3.8.

In some embodiments, the fSARA comprises a change from baseline in the fSARA total score of at least −4.0.

In some embodiments, the clinical assessment is the mDRS.

In some embodiments, the clinical assessment is the NPC-CSS.

In some embodiments, the clinical assessment is the 5-Domain NPC-CSS.

In some embodiments, the clinical assessment is the SCAFI.

In some embodiments, the clinical assessment is the 8MWT.

In some embodiments, the clinical assessment is the 9HPT.

In some embodiments, administering N-acetyl-DL-leucine or N-acetyl-L-leucine to the subject results in an improvement over baseline in at least one quality of life (QOL) assessment.

In some embodiments, the QOL assessment comprises the Clinical Global Impression of Severity and Improvement Scale (CGI-I), the EuroQol (EQ) 5Q-5D-5L/Y questionnaire, and/or the Visual Analogue Scale (VAS).

In some embodiments, the QOL assessment is the CGI-I.

In some embodiments, the QOL assessment is the EuroQol (EQ) 5Q-5D-5L/Y questionnaire.

In some embodiments, the QOL assessment is the VAS.

In some embodiments, N-acetyl-DL-leucine or N-acetyl-L-leucine is administered to the subject for at least 1 week.

In some embodiments, N-acetyl-DL-leucine or N-acetyl-L-leucine is administered to the subject for at least 4 weeks.

In some embodiments, N-acetyl-DL-leucine or N-acetyl-L-leucine is administered to the subject for at least 8 weeks.

In some embodiments, N-acetyl-DL-leucine or N-acetyl-L-leucine is administered to the subject for at least 12 weeks.

In some embodiments, N-acetyl-DL-leucine or N-acetyl-L-leucine is administered to the subject for at least 16 weeks.

In some embodiments, N-acetyl-DL-leucine or N-acetyl-L-leucine is administered to the subject for at least 20 weeks.

In some embodiments, N-acetyl-DL-leucine or N-acetyl-L-leucine is administered to the subject for at least 24 weeks.

In some embodiments, N-acetyl-DL-leucine or N-acetyl-L-leucine is administered to the subject for at least 28 weeks.

In some embodiments, N-acetyl-DL-leucine or N-acetyl-L-leucine is administered to the subject for at least 32 weeks.

A “therapeutically effective amount” of N-acetyl leucine is any amount which, when administered to a subject, is the amount that is needed to produce the desired effect, which, for the present disclosure, can be therapeutic and/or prophylactic. The dose may be determined according to various parameters, such as the N-acetyl leucine used, e.g., N-acetyl-DL-leucine or N-acetyl-L-leucine; the age, weight and condition of the patient to be treated; the route of administration; and the required regimen. A physician will be able to determine the required route of administration and dosage for any particular patient. For example, a total daily dose may be from about 0.1 g to about 30 g, about 0.7 g to about 15 g, from about 1 g to about 15 g, or from about 3 g to about 10 g of N-acetyl leucine may be administered, e.g., as a tablet or an oral suspension. In some embodiments, a total daily dose of about 0.7 g to about 4 g of N-acetyl-L-leucine is administered to a patient.

As used herein, “treating” or “treatment” refers to any indicia of success in arresting or ameliorating NPC in a subject, and/or arresting or ameliorating any one or more symptoms of NPC in a subject, including any objective or subjective parameter such as abatement; remission; diminishing, inhibiting, or eliminating one or more symptoms; making NPC more tolerable to the subject; slowing in the worsening of NPC; or improving the physical or mental well-being of the subject in need thereof.

The terms “treating” or “treatment” also encompasses, e.g., inducing inhibition, regression, rescue, or stasis of NPC. For example, treatment of a subject in need of treatment for NPC includes reducing a neurological symptom of NPC in the subject, inducing clinical response, inhibiting or reducing progression of NPC, or inhibiting or reducing a complication of NPC.

Preventing, arresting, or ameliorating NPC, such as preventing, diminishing, inhibiting, or eliminating one or more neurological symptoms of NPC can be based on objective and/or subjective parameters, including, e.g., the results of genetic testing, physical examination(s), neurological examination(s), and/or psychiatric evaluation(s). The success of treatment for NPC may be measured or evaluated by, for example, comparing the severity of NPC, or symptom thereof, before treatment with N-acetyl leucine is initiated, with the severity of NPC, or symptom thereof, following treatment with N-acetyl leucine. For example, the severity of NPC, or symptom thereof, may be assessed using a scale, index, rating, or score. In one embodiment, the treatment described herein improves such an assessment from a value or degree characteristic of a symptomatic subject to a value or degree characteristic of a non-symptomatic subject. In one embodiment, the treatment described herein improves such an assessment compared to a baseline. The baseline may be, for example, the subject's condition before initiating any treatment for NPC, or symptom thereof, or before initiating treatment for NPC, or symptom thereof, with N-acetyl leucine. Alternatively, the baseline may be, for example, the subject's condition after a certain time period on treatment for the disease. In one embodiment, treatment with N-acetyl leucine as described herein improves the subject's assessment, e.g., scale, index, rating, or score of objective and/or subjective parameters, compared to a baseline by at least 5%, at least 10%, at least 20%, at least 30%, at least 40%, or at least 50%. In one embodiment, assessment is improved by at least 60%, at least 70%, at least 80%, at least 90%, or 100%.

A “neurological symptom” or “neurological manifestation” of NPC includes any clinical or laboratory manifestation associated with NPC and is not limited to what the subject can feel or observe. Neurological symptoms of NPC include, but are not limited to, any one or more of the neurological symptoms listed in Table 2 and/or Table 6.

In another embodiment, the disclosure provides N-acetyl-L-leucine for the treatment of neurological manifestations of NPC in adult subjects. In another embodiment, the disclosure provides N-acetyl-L-leucine for the treatment of neurological manifestations of NPC in pediatric subjects weighing 15 kg or more. In some embodiments, the adult or pediatric subject has one or more NPC1 gene mutations listed in Table 1.

In another embodiment, the disclosure provides procedures of personalized medicine for subjects having NPC that encompasses the selection of treatment options with the highest likelihood of successful outcome for individual subjects, e.g., based on NPC1 gene mutations present in the subject. In another aspect, the disclosure relates to the use of a genetic testing to predict treatment outcome, e.g., the likelihood of favorable responses or treatment success, in subjects having NPC.

In another embodiment, the disclosure provides a method of selecting a subject for treatment of NPC with N-acetyl leucine, comprising obtaining a biological sample, e.g., a blood or saliva sample, from the subject, and testing the biological sample from the subject for the presence of a pathogenic variant, i.e., a mutation, in the NPC1 gene. Mutations in the NPC1 gene can be determined by genetic testing, e.g., chromosomal microarray analysis (CMA), single gene sequencing, multigene panel testing, and/or exome sequencing. The diagnosis of NPC may be confirmed by identification of either a pathogenic variant in NPC1. This selection method may further comprise administering a therapeutically effective amount of N-acetyl leucine to the subject if the biological sample contains a pathogenic variant in the NPC1 gene.

In another embodiment, the disclosure provides methods for predicting treatment outcomes in a subject having NPC, comprising obtaining a biological sample from the subject, testing the biological sample from the subject for the presence of a pathogenic variant in the NPC1 gene, wherein the detection of the pathogenic variant in the NPC1 gene indicates the subject will respond favorably to administration of a therapeutically effective amount of N-acetyl leucine. Favorable responses include, but are not limited to, preventing or delaying the onset of neurological symptoms that would normally be expected in a subject afflicted with NPC.

In another embodiment, the disclosure provides methods of treating a NPC in a subject, comprising administering a therapeutically effective amount of N-acetyl leucine to the subject wherein the subject's cells contain a pathogenic variant in the NPC1 gene. In one embodiment, the subject is selected for treatment with N-acetyl leucine after the subject's cells have been determined to contain a pathogenic variant in the NPC1 gene.

In another embodiment, the method of treating a subject having NPC comprises obtaining a biological sample from the subject, determining whether the biological sample contains a pathogenic variant in the NPC1 gene, and administering to the subject a therapeutically effective amount of N-acetyl leucine if the biological sample contains an elevated level or decreased level of the biomarker.

The term “biological sample” as used herein refers to any tissue or fluid from a subject that is suitable for detecting a pathogenic variant in the NPC1 gene. Examples of useful biological samples include, but are not limited to, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarker expression and/or mutation using any technique known in the art and can be obtained using techniques that are well within the scope of ordinary knowledge of a clinical practitioner. In one embodiment of the disclosure, the biological sample comprises blood cells.

The present disclosure provides the following particular embodiments with respect to personalized medicine for subjects having NPC:

Embodiment I: A method of treating a subject having NPC, the method comprising administering a therapeutically effective amount of N-acetyl leucine to the subject, wherein cells of the subject contain a pathogenic variant in the NPC1 gene.

Embodiment II: A method of treating a subject having NPC, the method comprising:
(a) determining whether a pathogenic variant in the NPC1 gene is present or absent in a biological sample taken from the subject; and
(b) administering a therapeutically effective amount of N-acetyl leucine to the subject if a pathogenic variant in the NPC1 gene is present in the biological sample.

Embodiment III: A method for treating a NPC in a subject having a pathogenic variant in the NPC1 gene, the method comprising administering to the subject a therapeutically effective amount of N-acetyl leucine.

Embodiment IV: A method of treating a subject having any one or more of the neurological symptoms of Table 2 and/or Table 6, the method comprising:
(a) determining whether a pathogenic variant in the NPC1 gene is present or absent in a biological sample taken from the subject; and
(b) administering a therapeutically effective amount of N-acetyl leucine to the subject if a pathogenic variant in the NPC1 gene is present in the biological sample.

Embodiment V: A method comprising administering a therapeutically effective amount of N-acetyl leucine to a subject in need thereof, wherein:
(a) the subject has any one or more of the neurological symptoms of Table 2 and/or Table 6; and
(b) a pathogenic variant in the NPC1 gene is present in a biological sample taken from the subject.

Embodiment VI. The method of any one of Embodiments I-V, wherein the N-acetyl leucine is N-acetyl-DL-leucine.

Embodiment VII. The method of any one of Embodiments I-V, wherein the N-acetyl leucine is N-acetyl-L-leucine.

Embodiment VII. The method of any one of Embodiments I-VIII, wherein the pathogenic variant in NPC1 is any one or more of the NPC1 gene mutations listed in Table 1.

In one embodiment, N-acetyl leucine may be administered, for example, at a dose ranging from about 700 mg to about 30 g per day or ranging from about 700 mg to about 15 g per day, such as ranging from about 1.5 g to about 10 g per day, optionally by solid oral or liquid oral route, e.g., as an oral suspension. N-acetyl leucine, may be administered, for example, in a dose according to that of Tanganil®, which is prescribed to adults in a dose of 1.5 g to 2 g per day, 3-4 tablets in two doses, morning and evening.

If a single enantiomer, e.g., N N-acetyl-L-leucine, is administered the doses may be adjusted accordingly. For instance if N-acetyl-L-leucine is administered, the dose may range from about 250 mg to about 30 g per day, range from about 250 mg to about 15 g per day, range from about 1 mg to about 10 g per day, or range from about 1 g to about 5 g per day.

In one embodiment, the administered total daily dose ranges of N-acetyl-leucine, e.g., N-acetyl-DL-leucine or N-acetyl-L-leucine, are from about 1 g to about 30 g per day, from about 1 g to about 15 g per day, from about 1 g to about 10 g per day, or from about 1.5 g to about 7 g per day, from 15.1 g to about 30 g per day, 16 g to about 30 g per day, 17 g to about 30 g per day, 18 g to about 30 g per day, 19 g to about 30 g per day, or 20 g to about 30 g per day. It may be from about 2, 3, 4, 5, 6, 7, 8, 9, 10, 11, 12, 13, or 14 g to about 15 g per day. It may be from about 2, 3, 4, 5, 6, 7, 8 or 9 g to about 10 g per day. It may be from 15.1, 16, 17, 18, 19, 20, 21, 22, 23, 24, 25, 25, 27, 28, or 29 g to about 30 g per day. It may be more than about 1.5 g per day, but less than about 15, 14, 13, 12, 11, 10, 9, 8, 7, 6 or 5 g per day. In one embodiment, the dose ranges from about 4 g to about 6 g per day. In one embodiment, the dose ranges from about 4 g to about 5 g per day. In one embodiment, the dose is about 4.5 g per day. In one embodiment, the dose is about 5 g per day. In one embodiment, the dose is about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, or about 15 g per day. In another embodiment, the dose is about 16 g per day, about 17 g per day, about 18 g per day, about 19 g per day, or about 20 g per day. In another embodiment, the dose is about 21 g per day, about 22 g per day, about 23 g per day, about 24 g per day, about 25 g per day, about 26 g per day, about 27 g per day, about 28 g per day, about 29 g per day, or about 30 g per day. In one embodiment, these doses are administered in a solid oral dosage form, notably tablets, or as an oral suspension. In another embodiment, these doses are for acetyl-leucine when in its racemic form. Doses for acetyl-leucine when an enantiomeric excess is present may or may not be lower, for example, around 50% lower. The above recited dose-ranges when halved are thus also explicitly encompassed by the present disclosure.

In one embodiment, the total daily dose of N-acetyl leucine may be spread across multiple administrations, i.e., administration of N-acetyl leucine may occur two, three, or more times a day to achieve the total daily dose. As an example, the required number of tablets or amount of an oral suspension to provide the total daily dose of N-acetyl-leucine may be split across two administrations (for example, in the morning and evening) or three administrations (for example, in the morning, noon, and evening). Each dose may be administered with or without food. For example, N-acetyl-L-leucine or N-acetyl-DL-leucine may be dosed by about 1 or about 2 hours before meals, such as at least about 20 minutes, at least about 30 minutes, at least about 40 minutes, or at least about 1 hour before meals, or may be dosed by about 1, about 2, or about 3 hours after meals, such as waiting at least about 20 minutes, at least about 30 minutes, at least about 1 hour, at least about 1.5 hours, at least about 2 hours, or at least about 2.5 hours after meals. For example, a total daily dose of 4.5 g acetyl-DL-leucine may be administered as three Tanganil® (or equivalent) tablets before, with, or after breakfast, three further tablets before, with, or after lunch and three further tablets before, with, or after dinner.

Treatment duration may be, for example, about seven days or more, about two weeks or more, about three weeks or more, about one month or more, about six weeks or more, about seven weeks or more, or about two months or more. In one embodiment, it is about three months or more, about four months or more, about five months or more or about six months or more. The treatment duration may be about 1 year or more, about 2 years or more, about 4 years or more, about 5 years or more, or about 10 years or more. The treatment duration may be the life-time of the subject.

Any and all combinations of dosage form, dose amount, dosing schedule and treatment duration are envisaged and encompassed by the disclosure. In one embodiment, the dose of N-acetyl-leucine is from about 4 g to about 15 g per day, taken across one, two, or three administrations per day, for a treatment duration of about two months or more. In another embodiment, the dose is more than 4 g but no more than 5 g per day, taken across one, two, or three administrations per day, for a treatment duration of about six months or more. The dosage form may be a solid oral dosage form, notably tablets. The dosage may also be in the form of an oral suspension.

N-acetyl-leucine may be used as a monotherapy for treating or preventing a NPC, or a neurological symptom thereof, in a subject. Alternatively, N-acetyl-leucine may be used as an adjunct to, or in combination with, other therapies for treating or preventing a NPC, or a neurological symptom thereof, in a subject. For example, N-acetyl-leucine may be used in combination with anti-seizure medications, e.g., valproate and lamotrigine, or miglustat to treat NPC.

In another embodiment, the disclosure provides a kit comprising N-acetyl-DL-leucine, or a pharmaceutically acceptable, and instructions for administering N-acetyl-DL-leucine, or a pharmaceutically acceptable to a subject having NPC, or a neurological symptom thereof, wherein the subject has one or more NPC1 gene mutations.

In another embodiment, the disclosure provides a kit comprising N-acetyl-L-leucine, or a pharmaceutically acceptable, and instructions for administering N-acetyl-L-leucine, or a pharmaceutically acceptable to a subject having NPC, or a neurological symptom thereof, wherein the subject has one or more NPC1 gene mutations.

All of the features described herein (including any accompanying claims, abstract and drawings), and/or all of the steps of any method so disclosed, may be combined with any of the above aspects in any combination, except combinations where at least some of such features and/or steps are mutually exclusive.

In another embodiment, the present disclosure provides a method of treating or preventing NPC, in a subject in need thereof, or treating or preventing a neurological symptom of NPC in a subject in need thereof, the method comprising administering about 0.7 g to about 30 g of N-acetyl-DL-leucine to the subject per day. In another embodiment, about 2 g to about 15 g of N-acetyl-DL-leucine is administered to the subject per day. In another embodiment, about 3 g to about 10 g of N-acetyl-DL-leucine is administered is administered to the subject per day. In another embodiment, about 4 g to about 8 g of N-acetyl-DL-leucine is administered to the subject per day. In another embodiment, about 4 g to about 5 g of N-acetyl-DL-leucine is administered to the subject per day. In another embodiment, about 15 g to about 30 g of N-acetyl-DL-leucine is administered to the subject per day. In another embodiment, about 5 g of N-acetyl-DL-leucine is administered to the subject per day.

In another embodiment, the present disclosure provides a method of treating or preventing NPC, in a subject in need thereof, or treating or preventing a neurological symptom of NPC in a subject in need thereof, the method comprising administering about 0.5 g to about 30 g of N-acetyl-L-leucine to the subject per day. In another embodiment, about 0.7 g to about 10 g of N-acetyl-L-leucine is administered to the subject per day. In another embodiment, about 0.7 g to about 5 g of N-acetyl-L-leucine is administered is administered to the subject per day. In another embodiment, about 0.7 g of N-acetyl-L-leucine is administered to the subject per day. In another embodiment, about 2 g of N-acetyl-L-leucine is administered to the subject per day. In another embodiment, about 3 g of N-acetyl-L-leucine is administered to the subject per day. In another embodiment, about 4 g of N-acetyl-L-leucine is administered to the subject per day. In another embodiment, about 5 g of N-acetyl-L-leucine is administered to the subject per day.

In some embodiments, N-acetyl-L-leucine is administered to the subject one, two, or three times per day according to the subject's body weight as set forth in Table A.

TABLE A

| Subject Body | Morning | | Afternoon | Evening |
|---|---|---|---|---|
| Weight | Dose | | Dose | Dose |
| 5 to <15 kg | 0.7 | g | No dose | No dose |
| 15 to <25 kg | 1 | g | No dose | 1 g |
| 25 to <35 kg | 1 | g | 1 g | 1 g |
| 35 kg or more | 2 | g | 1 g | 1 g |

The disclosure also provides the following particular method of treatment embodiments.

Embodiment 1. A method of treating or preventing Niemann-Pick disease type C (NPC), or a neurological symptom thereof in a subject in need thereof, the method comprising administering a therapeutically effective amount of N-acetyl-DL-leucine, or a pharmaceutically acceptable salt thereof, or N-acetyl-L-leucine, or a pharmaceutically acceptable salt thereof, to the subject, wherein the subject has any one or more of the NPC1 gene mutations provided in Table 1.

Embodiment 2. The method of Embodiment 1, wherein a first neurological symptom of NPC in the subject is any one or more of the neurological symptoms listed in Table 2.

Embodiment 3. The method of Embodiments 1 or 2, wherein the subject is less than two years old.

Embodiment 4. The method of Embodiments 1 or 2, wherein the subject is two years old to less than six years old.

Embodiment 5. The method of Embodiments 1 or 2, wherein the subject is six years old to less than 15 years old.

Embodiment 6. The method of Embodiments 1 or 2, wherein the subject is 15 years old or more.

Embodiment 7. The method of any one of Embodiments 1-6, wherein administering N-acetyl-DL-leucine, or N-acetyl-L-leucine to the subject results in an improvement over baseline in at least one clinical assessment used to measure the severity of neurological signs and symptoms of NPC.

Embodiment 8. The method of Embodiment 7, wherein the at least one clinical assessment is the Scale for the Assessment and Rating of Ataxia (SARA), the modified SARA (mSARA), the functional SARA (fSARA), the modified Disability Rating Scale (mDRS), Niemann-Pick type C Clinical Severity Scale (NPC-CSS), 5-Domain NPC-CSS, and/or the Spinocerebellar Ataxia Functional Index (SCAFI).

Embodiment 9. The method of Embodiment 8, wherein the clinical assessment is SARA.

Embodiment 10. The method of Embodiment 9, wherein SARA comprises a change from baseline in the SARA total score of at least −0.6.

Embodiment 11. The method of Embodiment 9, wherein SARA comprises a change from baseline in the SARA total score of at least −0.8.

Embodiment 12. The method of Embodiment 9, wherein SARA comprises a change from baseline in the SARA total score of at least −1.0.

Embodiment 13. The method of Embodiment 9, wherein SARA comprises a change from baseline in the SARA total score of at least −1.2.

Embodiment 14. The method of Embodiment 9, wherein SARA comprises a change from baseline in the SARA total score of at least −1.4.

Embodiment 15. The method of Embodiment 9, wherein SARA comprises a change from baseline in the SARA total score of at least −1.6

Embodiment 16. The method of Embodiment 9, wherein SARA comprises a change from baseline in the SARA total score of at least −1.8.

Embodiment 17. The method of Embodiment 9, wherein SARA comprises a change from baseline in the SARA total score of at least −2.0.

Embodiment 18. The method of Embodiment 8, wherein the clinical assessment is fSARA.

Embodiment 19. The method of Embodiment 18, wherein fSARA comprises a change from baseline in the fSARA total score of at least −0.2.

Embodiment 20. The method of Embodiment 18, wherein fSARA comprises a change from baseline in the fSARA total score of at least −0.3.

Embodiment 21 The method of Embodiment 18, wherein fSARA comprises a change from baseline in the fSARA total score of at least −0.4.

Embodiment 22. The method of Embodiment 18, wherein fSARA comprises a change from baseline in the fSARA total score of at least −0.5.

Embodiment 23. The method of Embodiment 18, wherein fSARA comprises a change from baseline in the fSARA total score of at least −0.6.

Embodiment 24. The method of Embodiment 18, wherein fSARA comprises a change from baseline in the fSARA total score of at least −0.7

Embodiment 25. The method of Embodiment 18, wherein fSARA comprises a change from baseline in the fSARA total score of at least −0.8.

Embodiment 26. The method of Embodiment 18, wherein fSARA comprises a change from baseline in the fSARA total score of at least −0.9.

Embodiment 27. The method of Embodiment 18, wherein fSARA comprises a change from baseline in the fSARA total score of at least −1.0.

Embodiment 28. The method of any one of Embodiments 1-27, wherein N-acetyl-DL-leucine or N-acetyl-L-leucine is administered to the subject for at least 1 week.

Embodiment 29. The method of any one of Embodiments 1-27, wherein N-acetyl-DL-leucine or N-acetyl-L-leucine is administered to the subject for at least 4 weeks.

Embodiment 30. The method of any one of Embodiments 1-27, wherein N-acetyl-DL-leucine or N-acetyl-L-leucine is administered to the subject for at least 8 weeks.

Embodiment 31. The method of any one of Embodiments 1-37, wherein N-acetyl-DL-leucine or N-acetyl-L-leucine is administered to the subject for at least 12 weeks.

Embodiment 32. The method of any one of Embodiments 1-31, wherein administering N-acetyl-DL-leucine or N-acetyl-L-leucine to the subject results in an improvement as compared to baseline in at least one quality of life (QOL) assessment.

Embodiment 33. The method of Embodiment 32, wherein the at least one QOL assessment is the Clinical Global Impression of Severity and Improvement Scale (CGI-I), the EuroQol (EQ) 5Q-5D-5L/Y questionnaire, and/or the Visual Analogue Scale (VAS).

Embodiment 34. The method of any one of Embodiments 1-33, wherein a total dose of about 0.5 g to about 30 g of N-acetyl-DL-leucine is administered to the subject per day.

Embodiment 35. The method of Embodiment 34, wherein a total dose of about 0.7 g to about 15 g of N-acetyl-DL-leucine is administered to the subject per day.

Embodiment 36. The method of Embodiment 35, wherein a total dose of about 1 g to about 10 g of N-acetyl-DL-leucine is administered to the subject per day.

Embodiment 37. The method of Embodiment 36, wherein a total dose of about 0.7 g to about 8 g of N-acetyl-DL-leucine is administered to the subject per day.

Embodiment 38. The method of Embodiment 37, wherein a total dose of about 0.7 g to about 5 g of N-acetyl-DL-leucine is administered to the subject per day.

Embodiment 39. The method of any one of Embodiments 34-38, wherein the N-acetyl-DL-leucine is administered once a day, twice a day, three times, or four times a day to the subject to achieve the total daily dose.

Embodiment 40. The method of any one of Embodiments 34-39, wherein the N-acetyl-DL-leucine is administered to the subject as a tablet or as an oral suspension.

Embodiment 41. The method of any one of Embodiments 1-33, wherein a total dose of about 0.5 g to about 30 g of N-acetyl-L-leucine is administered to the subject per day

Embodiment 42. The method of Embodiment 41, wherein a total dose of about 0.7 g to about 15 g of N-acetyl-L-leucine is administered to the subject per day.

Embodiment 43. The method of Embodiment 42, wherein a total dose of about 0.7 g to about 10 g of N-acetyl-L-leucine is administered to the subject per day.

Embodiment 44. The method of Embodiment 43, wherein a total dose of about 0.7 g to about 8 g of N-acetyl-L-leucine is administered to the subject per day.

Embodiment 45. The method of Embodiment 44, wherein a total dose of about 0.7 g to about 5 g of N-acetyl-L-leucine is administered to the subject per day.

Embodiment 46. The method of any one of Embodiments 41-45, wherein the N-acetyl-L-leucine is administered once a day, twice a day, three times, or four times a day to the subject to achieve the total daily dose.

Embodiment 47. The method of any one of Embodiments 41-46, wherein the N-acetyl-L-leucine is administered to the subject as a tablet or as an oral suspension.

Embodiment 48. The method of any one of Embodiments 1-47, where the subject has one or more homozygous NPC1 gene mutations.

Embodiment 49. The method of any one of Embodiments 1-47, where the subject has one or more heterozygous NPC1 gene mutations

Embodiment 50. The method of any one of Embodiments 1-49, wherein the subject has one of the NPC1 gene mutations provided in Table 1.

Embodiment 51. The method of any one of Embodiments 1-49, wherein the subject has two of the NPC1 gene mutations provided in Table 1.

Embodiment 52. The method of any one of Embodiments 1-49, wherein the subject has three of the NPC1 gene mutations provided in Table 1.

Embodiment 53. The method of any one of Embodiments 1-49, wherein the subject has four of the NPC1 gene mutations provided in Table 1.

Embodiment 54. The method of any one of Embodiments 1-40 or 48-53, wherein the N-acetyl-DL-leucine is administered to the subject in combination with one or more additional therapeutic agents.

Embodiment 55. The method of any one of Embodiments 1-33 or 41-53, wherein the N-acetyl-L-leucine is administered to the subject in combination with one or more additional therapeutic agents.

Embodiment 56. The method of Embodiments 54 or 55, wherein the one or more additional therapeutic agents comprise miglustat, levetiracetam, valproate, sertraline, donezepil, clonazepam, risperidone, melatonin, ramipril, metronidazole, lamotrigine, piracetam, 2-hydroxypropyl-β-cyclodextrin (HPBCD), vorinostat, lovastatin, rapamycin, and/or arimoclomol.

Embodiment 57. The method of Embodiment 56, wherein the one or more additional therapeutic agents comprise miglustat.

Embodiment 58. A kit for carrying out the method of any one of Embodiments 1-40, 48-54, 56, or 57, the kit comprising N-acetyl-DL-leucine and instructions for administering N-acetyl-DL-leucine to the subject.

Embodiment 59. A kit for carrying out the method of any one of Embodiments 1-33, 41-53, or 55-57, the kit comprising N-acetyl-L-leucine and instructions for administering N-acetyl-L-leucine to the subject.

Embodiment 60. A method of treating a subject having NPC, or a neurological symptom thereof, the method comprising:
(a) determining whether any one or more of the NPC1 gene mutations listed in Table 1 are present or absent in a biological sample taken from the subject; and
(b) administering a therapeutically effective amount of N-acetyl-DL-leucine, or a pharmaceutically acceptable salt thereof, or N-acetyl-L-leucine, or a pharmaceutically acceptable salt thereof, to the subject if any one or more of the NPC1 gene mutations are present in the biological sample.

Embodiment 61. A method, comprising administering a therapeutically effective amount of N-acetyl-DL-leucine, or a pharmaceutically acceptable salt thereof, or N-acetyl-L-leucine, or a pharmaceutically acceptable salt thereof, to a subject in need thereof, wherein:
(a) the subject has NPC, or a neurological symptom thereof; and
(b) any one or more of the NPC1 gene mutations listed in Table 1 is present in a biological sample taken from the subject.

### EXAMPLES

### Example 1

### Human Clinical Trial Data

Patients eligible for this study included patients aged ≥4 years with a diagnosis of NPC with clinical symptoms and signs referable to NPC. See Bremova-Ertl et al., N Engl J Med (2024) 390:421-31; DOI: 10.1056/NEJMoa2310151. All patients provided informed consent (or responsible person) and undertook a washout of any prohibited medications (e.g., N-acetyl-DL-leucine, N-acetyl-L-leucine, Sulfasalazine, Rosuvastatin) for 42 days before screening. The SARA score had to be between 7 and 34 (range 0-40 points; lower score representing better neurological status), indicating mild to severe symptoms. The eligibility criteria are given in Table 3.

TABLE 3
Inclusion Criteria

Individuals who meet all of the following criteria are eligible to participate in the
study:

| | |
|---|---|
| 1. | Written informed consent signed by the patient and/or their legal representative/ |
| | parent/impartial witness |
| 2. | Non-US: Male or female aged ≥4 years with a confirmed diagnosis of NPC at the time |
| | of signing informed consent. Confirmed diagnosis includes one of the following: |
| | a) Clinical features and positive biomarker screen and/or filipin test without |
| | genetic tests results (has not been performed) |
| | b) Clinical features and positive genetic test |
| | c) Clinical features and positive biomarker screen and/or filipin test but only one |
| | NPC mutation identified on genetic test |
| | d) Clinical features with positive biomarker screen and/or filipin test and positive |
| | genetic test |
| | US: Male or female aged ≥4 years with a confirmed genetic diagnosis of NPC at the |
| | time of signing informed consent. Patients must have clinical features of NPC and a |
| | positive genetic test for mutations in both copies of NPC1 or in both copies of NPC2. |
| 3. | Females of childbearing potential, defined as a premenopausal female capable of |
| | becoming pregnant, will be included if they are either sexually inactive (sexually |
| | abstinent for 14 days prior to the first dose and confirm to continue through 28 days |
| | after the last dose) or using one of the following highly effective contraceptives (i.e. |
| | results in <1% failure rate when used consistently and correctly) 14 days prior to the |
| | first dose continuing through 28 days after the dose: |
| | a) intrauterine device (IUD); |
| | b) surgical sterilization of the partner (vasectomy for 6 months minimum); |
| | c) combined (estrogen or progestogen containing) hormonal contraception |
| | associated with the inhibition of ovulation (either oral, intravaginal, or |
| | transdermal); |
| | d) progestogen only hormonal contraception associated with the inhibition of |
| | ovulation (either oral, injectable, or implantable); |
| | e) intrauterine hormone releasing system (IUS); |
| | f) bilateral tubal occlusion. |
| 4. | Females of non-childbearing potential who have undergone one of the following |
| | sterilization procedures at least 6 months prior to the first dose: |
| | a) hysteroscopic sterilization; |
| | b) bilateral salpingectomy; |
| | c) hysterectomy; |
| | d) bilateral oophorectomy; |

OR

| | |
|---|---|
| 5. | be postmenopausal with amenorrhea for at least 1 year prior to the first dose and |
| | follicle stimulating hormone (FSH) serum levels consistent with postmenopausal |
| | status. FSH analysis for postmenopausal women will be done at screening. FSH levels |
| | should be in the postmenopausal range as determined by the central laboratory. |
| | Non-vasectomized male patient agrees to use a condom with spermicide or abstain |
| | from sexual intercourse during the study until 90 days beyond the last dose of study |
| | medication and the female partner agrees to comply with inclusion criteria 3 or 4. For |
| | a vasectomized male who has had his vasectomy 6 months or more prior to study start, |
| | it is required that they use a condom during sexual intercourse. A male who has been |
| | vasectomized less than 6 months prior to study start must follow the same restrictions |
| | as a nonvasectomized male. |
| 6. | If male, patient agrees not to donate sperm from the first dose until 90 days after their |
| | last dose. |
| 7. | Patients must fall within: |
| | a) A SARA score of 7 ≤ X ≤ 34 points (out of 40) |

AND

```
  b) Either:
    i. Within the 2-7 range (0-8 range) of the Gait subtest of the SARA scale
```

OR

| | |
|---|---|
| | ii. Be able to perform the 9-Hole Peg Test with Dominant Hand (9HPT-D) |
| | (SCAFI subtest) in 20 ≤ X ≤ 150 seconds. |
| 8. | Weight ≥15 kg at screening. |
| 9. | Patients are willing to disclose their existing medications/therapies for (the symptoms) |
| | of NPC, including those on the prohibited medication list. Non-prohibited |
| | medications/therapies (authorized medicines for NPC [e.g. miglustat], speech therapy, |
| | and physiotherapy are permitted provided: |
| | a) The Investigator does not believe the medication/therapy will interfere with the |
| | study protocol/results |
| | b) Patients have been on a stable dose/duration and type of therapy for at least 42 |
| | days before Visit 1 (Baseline 1) |
| | c) Patients are willing to maintain a stable dose/do not change their therapy |
| | throughout the duration of the study. |
| 10. | An understanding of the implications of study participation, provided in the written |
| | patient information and informed consent by patients or their legal |
| | representative/parent, and demonstrates a willingness to comply with instructions and |
| | attend required study visits (for children this criterion will also be assessed in parents |
| | or appointed guardians). |

Exclusion Criteria

Individuals who meet any of the following criteria are not eligible to participate in the study:

| | |
|---|---|
| 1. | Patients who have any known hypersensitivity or history of hypersensitivity to: |
| | a) Acetyl-Leucine (DL-, L-, D-) or derivatives. |
| | b) Excipients the IB1001 sachet (namely isomalt, hypromellose, and strawberry |
| | flavour). |
| | c) Excipients the placebo sachet (namely isomalt, hypromellose, strawberry |
| | flavour, citric acid, microcrystalline cellulose, lactose, denatonium benzoate). |
| 2. | Simultaneous participation in another clinical study or participation in any clinical |
| | study involving administration of an investigational medicinal product (IMP; ‘study |
| | drug’) for at least 42 days prior to Visit 1. At the discretion of the investigator, Medical |
| | Monitor, and Sponsor, the washout period for specific IMPs may be longer based on |
| | the pharmacological activity and pharmacokinetics of the drug. |
| 3. | Patients with a physical or psychiatric condition which, at the investigator's discretion |
| | and in consultation with the Medical Monitor and Sponsor (as applicable), may put the |
| | patient at risk, may confound the study results, or may interfere with the patient's |
| | participation in the clinical study, i.e. reliably perform study assessments. |
| 4. | Known or persistent use, misuse, or dependency of medication, drugs, or alcohol. |
| 5. | Current or planned pregnancy or women who are breastfeeding. |
| 6. | Patients with severe vision or hearing impairment (that is not corrected by glasses or |
| | hearing aids) that, at the investigator's discretion, interferes with their ability to |
| | perform study assessments. |
| 7. | Patients who have been diagnosed with arthritis or other musculoskeletal disorders |
| | affecting joints, muscles, ligaments, and/or nerves that by themselves affects patient's |
| | mobility and, at the investigator's discretion, interferes with their ability to perform |
| | study assessments. |
| 8. | Patients unwilling and/or not able to undergo a 42-day washout period from any of the |
| | following prohibited medication prior to Visit 1 (Baseline 1) and remain without |
| | prohibited medication through Visit 6. |
| | a) N-Acetyl-DL-Leucine (e.g. Tanganil ®); |
| | b) N-Acetyl-L-Leucine; |
| | c) Sulfasalazine; |
| | d) Rosuvastatin. |

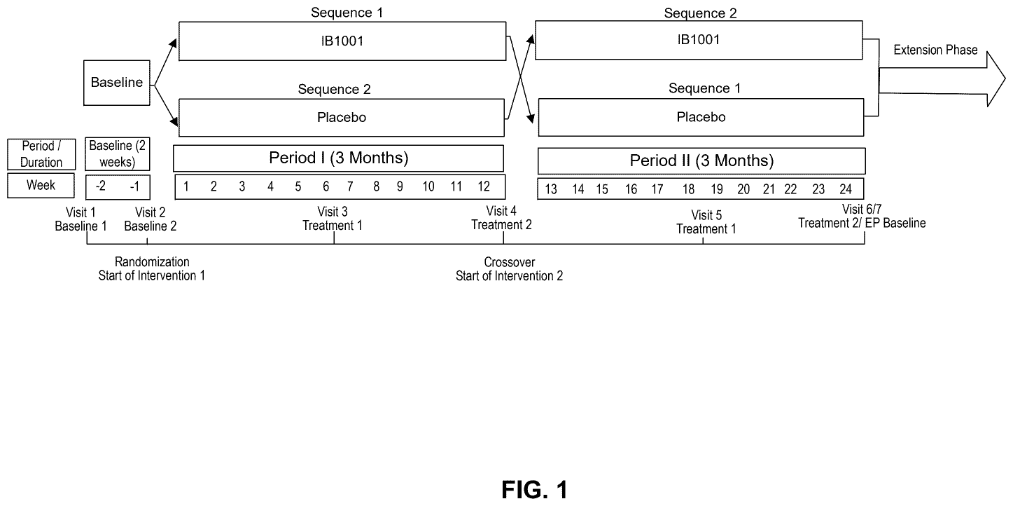

The trial consisted of a baseline period followed by two 12-week consecutive treatment periods (FIG. 1). Two visits (Visit 1 and Visit 2) were conducted in the baseline period, which occurred 14-21 days apart. Safety and efficacy assessments were performed at both visits. At the second baseline visit (Visit 2), eligible patients were randomized using computerized interactive response technology in a 1:1 ratio to receive either NALL (also referred to as IB1001) or placebo in Period I, which they immediately commenced and received for 84-91 days. At the end of the first treatment period (Visit 4 at 12 weeks), patients immediately crossed over to receive the opposite trial drug in Period II for 84-91 days. Two visits occurred in each period (Visit 3 and 4 in Period I; and Visit 5 and 6 in Period II:). Patients, their families/caregivers, the Investigators/trial teams and the Sponsor were masked to treatment allocations until after the database was locked. Patients who completed the trial (Visit 6) were eligible to continue into an open-label, long-term extension phase, which is ongoing and is a component of the original protocol.

During the active treatment periods, patients aged ≥13 years or aged 4-12 years weighing ≥35 kg received 4 g/day of orally administered NALL or matching placebo developed to have the same color, taste, appearance, and solubility properties to the active agent (granules in a sachet for suspension in 40 mL water, orange juice, or almond milk) three times per day (2 g in the morning, 1 g in the afternoon, and 1 g in the evening). Patients aged 4-12 years weighing <35 kg received weight-tiered doses two or three times per day based on an approximate total dose of 0.1 g/kg/day.

The primary endpoint prespecified in the original protocol in all jurisdictions but the US was the Scale for the Assessment and Rating of Ataxia (SARA), an eight-item (Gait, Stance, Sitting, Speech disturbance, Finger chase, Nose-finger test, Fast alternating hand movement, Heel-shin slide) clinical rating scale ranging from 0-40, where 0 is the best neurological status and 40 the worst. It is a reliable and validated clinical scale with high internal consistency (in spinocerebellar ataxias but has not been validated in NPC) that measures the severity of neurological signs and symptoms. These domains are functional do not represent an isolated measure of cerebellar ataxia, but reflect the various neurological systems which are impaired in NPC and lead to functional decline. At the request of the US Food and Drug Administration, a modified SARA (mSARA), which excluded the Sitting and Stance domains (range 0 to 30 points), was the US primary endpoint. In this disclosure, the SARA is reported as the endpoint for primary analysis and mSARA is considered ancillary and reported without a p-value for differences between groups.

Secondary endpoints were the modified Disability Rating Scale (mDRS), consisting of 6 subdomains (Ambulation, Manipulation, Seizures, Language, Swallowing, Ocular movements) total ranging from 0-24, which evaluated the overall neurological status, in which 0 is the best neurological status and 24 is the worst. Cerebellar function was further assessed using the Spinocerebellar Ataxia Functional Index (SCAFI), comprising the timed 8-Meter Walk Test (8MWT), the timed 9-Hole Peg Test (9HPT) with the dominant and non-dominant hand, and the number of spoken “PATA” repetitions within 10 seconds. Each test was carried out twice and values were averaged; the 8MWT and 9HPT values were converted from times to rates, and the results expressed as a composite Z-score of each test relative to baseline. Subjective impairment and quality of life were evaluated using the Clinical Global Impression of Severity and Improvement Scale (CGI-I) completed by the Investigators,

Caregivers, and Patients (a 7-point Likert scale (−3 to 3) where-3 is very much improved, 0 is no change, and 3 is very much worse) the EuroQol (EQ) 5Q-5D-5L/Y questionnaire and the Visual Analogue Scale (VAS) and exit interviews. The NPC Clinical Severity Scale was an exploratory measure, ranging from 0-54, where 0 is the best neurological status and 54 is the worst. This scale was developed and validated for clinical assessment of NPC disease progression for a minimum of one-year.

Safety assessments included adverse event (AE) monitoring, for which the relation of the trial agent of each AE was assessed by the site investigator or their delegates, clinical laboratory testing and limited pharmacokinetic sampling, physical examinations, vital signs, and electrocardiograms.

A sample size of 46 patients was estimated to provide 80% power, at a one-sided significance level of 5%, to detect a mean difference between groups in the SARA and mSARA scores of 1.0 and 0.85 (respectively), and a standard deviation for the total SARA and mSARA scores between 7.5 and 8.5 and 6.375 and 7.225 (respectively) based on analysis of covariance with the baseline SARA or mSARA score at the start of Period I as the covariate.

The primary efficacy analyses for both SARA and mSARA was analysis of covariance model with the difference between the SARA/mSARA outcomes at visits 4 and 6 as the dependent variable, with baseline SARA/mSARA score and an indicator for sequence as the independent variables. The estimated coefficient of the indicator for sequence in this model provides the least squares (LS) estimate of the difference in the treatment means on division by 2. This method of analysis accounts for any cross-over period effects and evaluates the within-patient differences. Statistical analysis was to be based on the modified Intention-to-Treat (mITT) analysis set, consisting of all randomized patients who received at least one dose of investigational product at Visit 2 (start of intervention). All patients received at least one dose so that this analysis set in this case corresponds the Intention-to-Treat population. One patient withdrew from the trial during period I and to accommodate this the model for analysis was implemented using a Mixed Model, assuming Missing at Random. As noted, SARA is used for the primary analysis to avoid issues related to multiple comparisons. Two-sided p-values of the null hypothesis were calculated and a significance level of 0.05 used to judge statistical significance.

The SCAFI and mDRS secondary endpoints were evaluated as per the primary endpoint; for the CGI-I the change from Baseline to Period I for the two treatment groups was assessed. For each primary and secondary endpoint, there were separate evaluations within key subgroups pre-defined in the Statistical Analysis Plan (the trial was not powered for conclusions in these subgroups). Because there was no prespecified plan for adjustment of the widths of confidence intervals for multiple comparisons, results for secondary endpoints are presented as point estimates and 95% confidence intervals without p values and no definite conclusions can be drawn from these data. SAS 9.4 (SAS Institute, Cary N.C.) was used for analysis. The Safety Analysis Set) consisted of all patients who received at least one dose of study drug.

Sixty four patients were screened, and 60 patients were enrolled (aged 5 to 67 years) and randomly assigned to a treatment group sequence. Four patients were excluded because their SARA scores were outside the range of 7 to 34. The demographic and baseline clinical characteristics of the enrolled patients are presented in Table 4; the genotype and additional information on the phenotype and clinical characteristics of each patient is presented in Table 6.

TABLE 4

| Parameter | Statistic/Category | Total (n = 60) | |
|---|---|---|---|
| Age | Paediatric (<18 years) | 23 | (38.3%) |
| | Adult (>=18 years) | 37 | (61.7%) |
| Gender (n (%)) | Female | 27 | (45.0%) |
| | Male | 33 | (55.0%) |
| Race (n (%)) | American Indian or Alaska native | 0 | (0.0%) |
| | Asian | 0 | (0.0%) |
| | Black or African American | 2 | (3.3%) |
| | Native Hawaiian or other Pacific Islander | 0 | (0.0%) |
| | White | 54 | (90.0%) |
| | Other | 4 | (6.7%) |
| Age at diagnosis group (n (%)) | Early-infantile (<2 years) | 9 | (15.0%) |
| | Late-infantile (2 to <6 years) | 14 | (23.3%) |
| | Juvenile (6 to <15 years) | 23 | (38.3%) |
| | Adolescent/Adult (>=15 years) | 14 | (23.3%) |
| Disease duration | Mean (SD) | 171.32 | (116.70) |
| (months)* | Median | 153.43 | |
| | Min, Max | 19.1, 514.3 | |
| Dose group (n (%)) | Age 4-12 years - 15 to <25 kg - 2 g per day | 6 | (10.0%) |
| | Age 4-12 years - 25 to <35 kg- 3 g per day | 3 | (5.0%) |
| | Age 4-12 years - >=35 kg - 4 g per day | 3 | (5.0%) |
| | Age >=13 years - 4 g per day | 48 | (80.0%) |
| Miglustat at | Yes | 51 | (85.0%) |
| baseline** (n (%)) | No | 9 | (15.0%) |

*Disease duration was calculated as the difference in days between the Visit 1 date and the date of disease start +1 and then converted to months. Disease start was defined as the earliest of 2 dates: start date of the Medical History item or the date of genetic confirmation of the disease. Partially missing dates were imputed using the first day of the month and the first month of the year in case month and/or day were missing.
**Indicates concurrent miglustat use throughout the duration of the trial

A total of 30 (50%) patients were assigned to Sequence 1 (NALL to Placebo) and 30 (50%) patients were assigned to Sequence 2 (Placebo to NALL). One patient in Sequence 1 (NALL to Placebo) was withdrawn in Period I between Visit 3 and Visit 4 due to a serious adverse event unrelated to trial treatment (complications during a pre-planned placement of a percutaneous endoscopic gastrostomy feeding tube leading to a prolonged hospitalization due to aspiration pneumonia, which was fatal). The patient's available data was used in the primary analysis set by using last observation carried forward. The baseline SARA was 14.90 (SD=7.49) for patients randomized in Period I (n=30) and 16.87 (SD=7.51) for patients randomized to placebo in period I (n-30). A total of 85% of patients had been treated with miglustat that continued through the trial. Other baseline score values are given in Table 5. A total of 59 patients (98.3%) completed the study.

TABLE 5

| | | | |
|---|---|---|---|
| Mean | | Mean | |
| Baseline | | Baseline | |
| NALL | n= | Placebo | n= |

Primary Endpoint

| | | | | |
|---|---|---|---|---|
| Scale for the Assessment and | 15.88 | 60 | 15.68 | 59 |
| Rating of Ataxia (SARA) | (SD = 7.50) | | (SD = 7.39) | |
| Modified Scale for the | 13.20 | 60 | 13.03 | 59 |
| Assessment and Rating of | (SD = 5.50) | | (SD = 5.39) | |
| Ataxia (mSARA) | | | | |

Secondary Endpoints

| | | | | |
|---|---|---|---|---|
| Investigator's Clinical Global | N/A | N/A | N/A | N/A |
| Impression of Improvement | | | | |
| Caregiver Clinical Global | N/A | N/A | N/A | N/A |
| Impression of Improvement | | | | |
| Patient Clinical Global | N/A | N/A | N/A | N/A |
| Impression of Improvement | | | | |
| Spinocerebellar Ataxia | −0.29 | 59 | −0.26 | 58 |
| Functional Index (SCAFI) | (SD = l.03) | | (SD = l.01) | |
| Modified Disability Rating | 0.480 | 60 | 0.477 | 59 |
| Scale (mDRS)* | (SD = 0.149) | | (SD = 0.149) | |

Exploratory Endpoint

| | | | | |
|---|---|---|---|---|
| Niemann-Pick Disease type C | 18.1 | 59 | 17.9 | 58 |
| Clinical Severity Scale | (SD = 7.1) | | (SD = 7.0) | |

*mDRS was scaled from 24 total points to a nought-to-one (0 to 1)

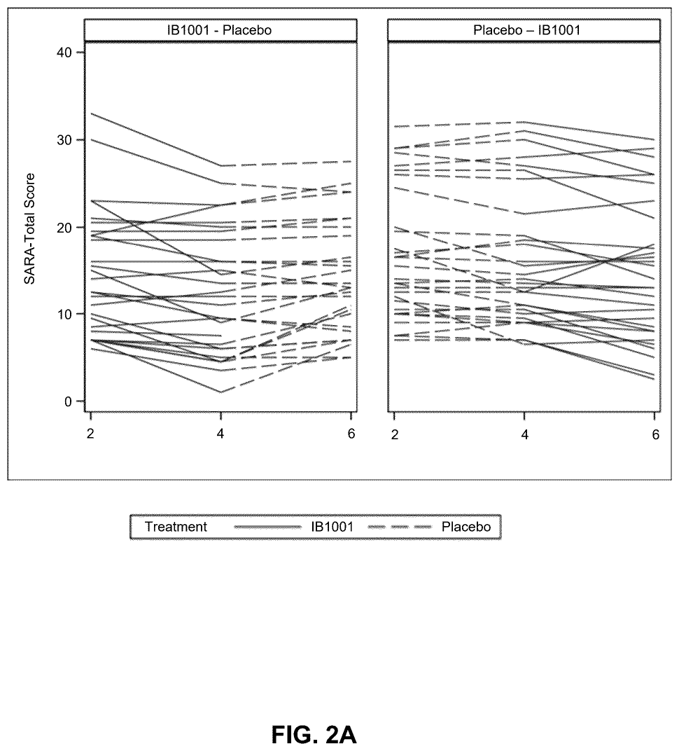

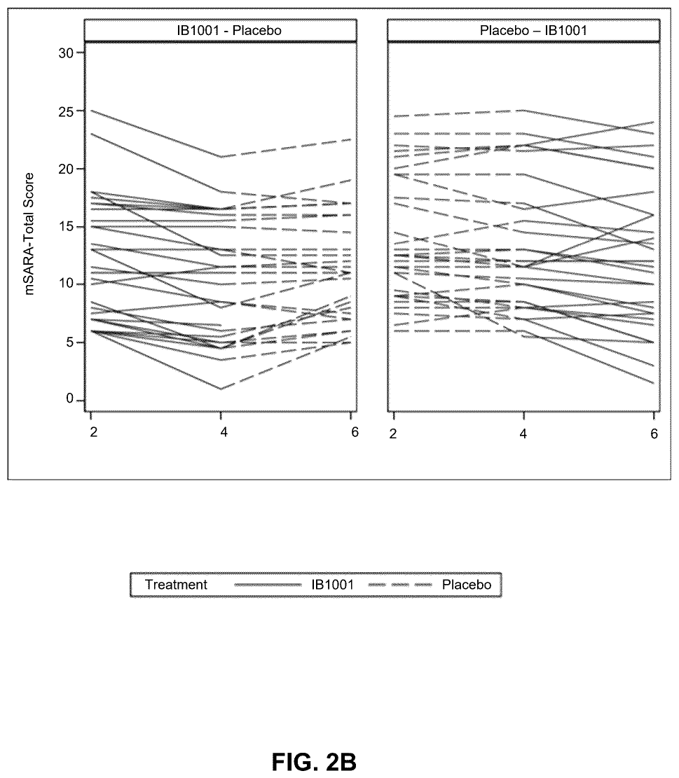

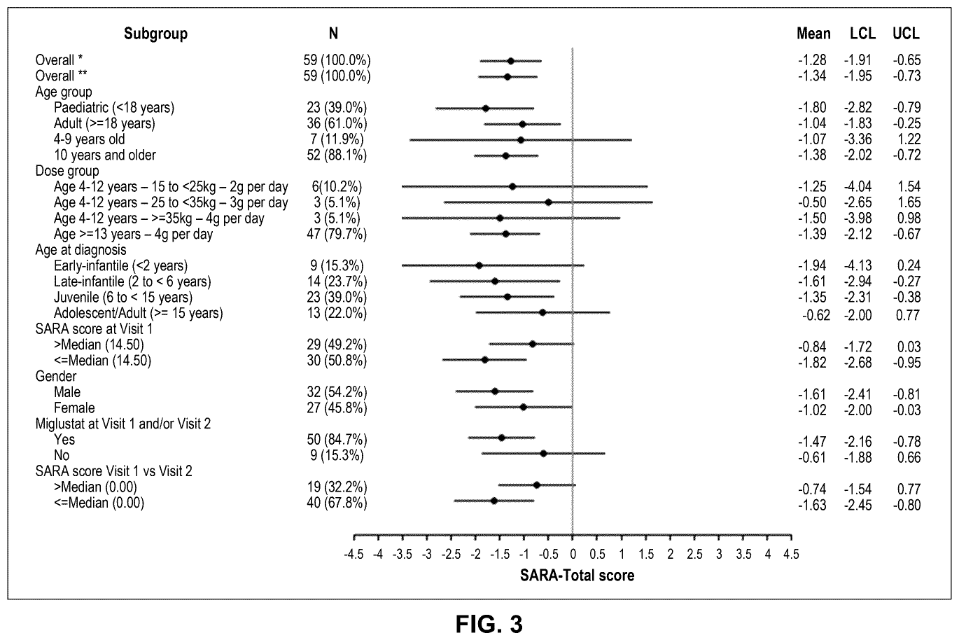

The mean change in the SARA was −1.97 (SD=2.43) with NALL treatment and −0.60 (SD=2.39) with placebo (LS mean difference −1.28; 95% Confidence Interval [CI], −1.91, to −0.65; p<0.001) (Table 7). The mean change on the mSARA score was similar to the SARA analysis. (Table 7). Spaghetti plots for individual patients' SARA and mSARA total scores versus visits per treatment sequence are provided in FIG. 2A and FIG. 2B, respectively. SARA subgroup analyses are shown in FIG. 3. The NALL vs. placebo difference for representative subjects is provided in Table 8.

TABLE 8

| | | | NALL−Placebo |
|---|---|---|---|
| Subject ID | Placebo | NALL | Difference |
| 31-1-01 | −0.5 | 0 | 0.5 |
| 31-1-02 | −0.5 | 0 | 0.5 |
| 31-1-04 | −1.5 | −1 | 0.5 |
| 31-1-06 | 1 | −1.5 | −2.5 |
| 31-1-07 | −2 | −2 | 0 |
| 31-1-08 | 0.5 | −1.5 | −2 |
| 31-1-09 | 0.5 | 0 | −0.5 |
| 31-1-10 | −0.5 | −0.5 | 0 |
| 31-1-11 | −0.5 | −1 | −0.5 |
| 41-1-01 | 0 | −1.5 | −1.5 |
| 41-1-02 | 0 | −4.5 | −4.5 |
| 41-1-03 | −4.5 | −3 | 1.5 |
| 41-1-04 | 3.5 | −2.5 | −6 |
| 41-1-05 | 1.5 | −5 | −6.5 |
| 41-1-07 | −2.5 | −5 | −2.5 |
| 41-1-08 | 3 | −0.5 | −3.5 |
| 41-1-09 | 2 | −0.5 | −2.5 |
| 41-1-10 | −3 | −3 | 0 |
| 41-1-11 | −0.5 | −5 | −4.5 |
| 41-1-12 | −0.5 | −6 | −5.5 |
| 41-1-13 | 0 | −4.5 | −4.5 |
| 42-1-01 | 0 | −1 | −1 |
| 42-1-02 | 0 | −1.5 | −1.5 |
| 43-1-01 | −2 | −2 | 0 |
| 43-1-02 | 2 | 1 | −1 |
| 43-1-03 | | −0.5 | |
| 43-1-04 | 0 | 1 | 1 |
| 44-1-01 | −1 | 1 | 2 |
| 44-1-02 | −1 | −2.5 | −1.5 |
| 44-3-01 | 0 | 0 | 0 |
| 44-3-02 | 0.5 | 0 | −0.5 |
| 44-3-03 | 0 | 0 | 0 |
| 44-3-04 | −1 | −1 | 0 |
| 44-4-01 | −6 | −5 | 1 |
| 44-4-02 | 5 | 3.5 | −1.5 |
| 44-4-03 | −1 | 1.5 | 2.5 |
| 44-4-04 | 0 | −1 | −1 |
| 49-3-01 | 1 | −2 | −3 |
| 49-4-01 | 0 | −5.5 | −5.5 |
| 49-4-02 | −0.5 | −5.5 | −5 |
| 49-4-03 | −2 | −6 | −4 |
| 49-4-04 | 1 | −3 | −4 |
| 49-4-05 | 0.5 | −1.5 | −2 |
| 49-4-06 | −3 | −4 | −1 |
| 49-4-08 | 2 | −1 | −3 |
| 49-4-09 | 0 | −2.5 | −2.5 |
| 49-5-01 | −6.5 | −8.5 | −2 |
| 49-5-02 | 1.5 | 0 | −1.5 |
| 49-5-03 | −5.5 | −5 | 0.5 |
| 49-5-04 | −4.5 | −3.5 | 1 |
| 49-5-06 | 4 | 1.5 | −2.5 |
| 49-5-07 | −3 | −1.5 | 1.5 |
| 49-5-08 | −5.5 | −6 | −0.5 |
| 61-1-01 | −1.5 | −3.5 | −2 |
| 61-1-02 | −0.5 | −4.5 | −4 |
| 61-1-03 | −5 | 0.5 | 5.5 |
| 61-1-04 | 1 | 2 | 1 |
| 61-1-05 | 1.5 | −1 | −2.5 |
| 61-1-06 | −1 | −0.5 | 0.5 |

In Period I, patients who received Placebo (n=30) had a change from baseline in the SARA of −0.60. Patients who received NALL in Period I followed by Placebo in Period II (n=29) had a worsening of symptoms on Placebo, (mean difference from Visit 4 [end of NALL treatment], +1.55 SARA), reflecting a deterioration in neurological signs and symptoms when treatment with NALL was stopped.

Secondary endpoints were generally in the same direction as the primary endpoint: CGI-I Investigators' (mean difference −0.6; 95% CI, −1.07, to −0.13), Caregivers' CGI (mean difference −0.7; 95% CI, −1.17, to −0.23), and Patients' CGI (mean difference −0.5; 95% CI, −1.10, to +0.10) scale, the mDRS (mean baseline NALL: 0.477 (SD-0.124); Placebo: 0.475 (SD-0.142); mean difference −0.029; 95% CI, −0.048, to −0.010) and SCAFI assessment (mean baseline NALL: −0.39 (SD=1.04); Placebo: −0.35 (SD=1.02); mean difference 0.7; 95% CI, −0.0, to 0.15) scales (Table 7). Quality of life changes measured by the EQ-5D are shown in the supplementary appendix.

The frequency of AEs for patients receiving NALL was (36 patients, 79 events) versus Placebo (30 patients, 75 events). No TEAEs led to premature discontinuation of the trial. No TEAEs occurred in more than 10% of patients on NALL. Three patients had 1 TEAE that were assessed as by the investigator as related to NALL (anal incontinence, restless legs, and rosacea). These events were all transient. The incidence of upper respiratory tract infection was higher when patients on treatment with NALL (10.0%) versus placebo (5.1%). The incidence of falls was lower when patients were on treatment with NALL (6.7%) versus placebo (15.3%).

No trial drug-related serious adverse events (SAEs) occurred. One death was attributed due to an unrelated SAE (aspiration pneumonia after a pre-planned percutaneous endoscopic gastrostomy tube). Results of plasma and urine tests, vital signs, and ECG recordings were normal or rated as clinically non-significant. Adherence to trial drug was high as shown by treatment compliance and the regular urine analyses for prohibited medication.

TABLE 6

| | | | Age at First | | | |
|---|---|---|---|---|---|---|
| Subject | | | Neurological | | First Neurological | Neurological Symptoms |
| ID | Genetic Diagnosis (genotype) | Phenotype** | Symptom* | | Symptom*** | at Screening*** |
| 31-1-01 | c.3019C > G p.(Pro1007Ala) AND | Early- | 4.5 | years | cognitive | cognitive impairment, behavioural |
| | c.1654 + 5G > A, p.(?) in NPC1 | infantile | | | impairment/decline | problems, supranuclear gaze palsy, mild |
| | | | | | | dysmetria |
| 31-1-02 | c.275A > G (p.Gln92Arg) and | Early- | 3 | years | cognitive decline | cognitive impairment, supranuclear gaze |
| | c.1918G > A (p.Gly640Arg) in NPC1 | infantile | | | and cerebellar ataxia | palsy, pyramidal signs, cerebellar ataxia, |
| | | | | | | gait impairment, mild dysmetria, |
| | | | | | | cataplexy |
| 31-1-04 | c.[1918G > A](p.[Gly640Arg](+) | Late-infantile | around 6 | | clumsiness/falling | cerebellar ataxia, supranuclear gaze palsy, |
| | [3451G > A]p.Ala1151Thr) in NPC1 | | years | | and ADHD | dysphagia, dysarhtria, mild dysmetria, |
| | | | | | | cognitive impairment, gait impairment |
| 31-1-06 | c.2474A > G(p.Tyr825Cys) AND | Adolescent/ | 52 | years | dystonia hands | supranuclear gaze palsy, cerebellar ataxia, |
| | c.3019C > G p.(Pro1007Ala) in NPC1 | Adult | | | | tremor, dysarthria, dysphagia, dysmetria, |
| | | | | | | cognitive decline |
| 31-1-07 | c.2861C > T, p.(Ser954Leu) and | Late-infantile | around 6 | | clumsiness | cerebellar ataxia, supranuclear gaze palsy, |
| | c.1574A > T, p.(asp525Val) in NPC1 | | years | | | dysphagia, mild dysmetria, cognitive |
| | | | | | | impairment, dysarthria, cognitive |
| | | | | | | impairment, dysmetria |
| 31-1-08 | c.2861C > T, p.(Ser954Leu) and | Adolescent/ | around 30 | | dystonia hands | dystonia, dysmetria, dysarthria, cerebellar |
| | c.1574A > T, p.(asp525Val) in NPC1 | Adult | years | | | ataxia |
| 31-1-09 | c.306T > G p.Tyr102* AND | Adolescent/ | around 50 | | cognitive decline, | cognitive impairment, supranuclear gaze |
| | c.180G > T, p.Gln60His | Adult | years | | supranuclear gaze | palsy, tremor, cerebella ataxia, dysphagia, |
| | | | | | palse | dysathria, dysmetria, depression |
| 31-1-10 | Homozygous c. 1918G > A | Juvenile | around 15 | | frequent | cognitive impairment, supranuclear gaze |
| | (p.Gly640Arg) in NPC1 | | years | | falling/tripping | palsy, cerebella ataxia, dysphagia, |
| | | | | | | dysathria, dysmetria |
| 31-1-11 | c.3451G > A, (p.Ala1151Thr) AND | Adolescent/ | around 40 | | dystonia | supranuclear gaze palsy, cerebellar ataxia, |
| | c.2474 A > G, p.(Tyr825Cys) | Adult | years | | | dysarthria, dysphagia, dysmetria, |
| | | | | | | dystonia, hearing loss |
| 41-1-01 | NPC1: c.2780C > T | Late-infantile | 5 | years | Ataxia | Spastic-ataxic gait with rare falls, discrete |
| | (p.A1a927Val)undc.1295delG | | | | | acral dystonia with intentional tremor, |
| | (p.Gly432Aspfs*17) | | | | | dysarthrophonia, facial dystonia, vertical |
| | | | | | | supranuclear saccade palsy (VSSP), |
| | | | | | | compensated by blinks and head thrusts. |
| | | | | | | Systemic disease: Splenomegaly |
| 41-1-02 | NPC1: Homozygous T3128 −> C | Adolescent/ | 15 | years | Ataxia | Spastic-ataxic gait, hand and facial |
| | (Ile1061 −> Thr) | Early | | | | dystonia, intentional tremor, discrete |
| | | adulthood | | | | dysphagia, dysartrhophonia, VSSP with |
| | | | | | | blinks and head thrusts, mild cognitive |
| | | | | | | decline with psychomotor slowing, mild |
| | | | | | | depression. |
| 41-1-03 | NPC1: p.I1061T and p.D948N | Early- | 1 | year | Ataxia | Spastic-ataxic gait with contractures of |
| | | infantile | | | | the hip extensors and knee flexor, foot |
| | | | | | | deformities, hand dystonia with acral |
| | | | | | | hypotonia, disdiadochokinesia dysphagia, |
| | | | | | | dyphonia, VSSP with blinks and head |
| | | | | | | thrusts, mild cognitive decline, well- |
| | | | | | | controlled epilepsy. Systemic disease: |
| | | | | | | severe splenomegaly |
| 41-1-04 | NPC1: Homozygous c.3246-5_3246- | Juvenile | 13 | years | Dysarthrophonia | Dystonia of the left foot, mild gait and |
| | 7del′ | | | | | stance ataxia with ffalls, |
| | | | | | | disdiadochokinesia, mild dysmetria, |
| | | | | | | nasolalia and dysarthrophonia, mild |
| | | | | | | cognitive decline, depression. Systemic |
| | | | | | | disease: Splenomegaly |
| 41-1-05 | NPC1: c.2819C < T(p.Ser940Leu) and | Juvenile | 12 | years | Dystonia | Mild stance and gait ataxia, acral |
| | c.2861C > T(p.Ser.954Leu) | | | | | dystonia, intentional tremor, VSSP. |
| | | | | | | Behavioural problems, ADHD and |
| | | | | | | depression. Systemic disease: |
| | | | | | | Splenomegaly |
| 41-1-07 | NPC1: c.2657G > T and c.2932C > T | Late-infantile | 5 | years | Epilepsy | Ataxic-dystonic gait with foot |
| | | | | | | deformities, disdiadochokinesia, |
| | | | | | | treatment-refractory epilepsy, dystonic |
| | | | | | | posturing, facial dystonia, vertical |
| | | | | | | supranuclear gaze palsy (VSGP), medium |
| | | | | | | cognitive decline with psychomotor |
| | | | | | | slowing. Systemic disease: |
| | | | | | | Hepatosplenomegaly. |
| 41-1-08 | NPC1: c.2692G > A and c.3773delCC | Adolescent/ | 18 | years | Cognitive decline | Discrete gait and stance ataxia, |
| | | Adult | | | | perpendicular ataxia, dysarthria. Moderate |
| | | | | | | to severe dementia with psychomotor |
| | | | | | | slowing. Systemic disease: Splenomegaly |
| | | | | | | with prior splenectomy due to a rupture |
| | | | | | | psychomotor slowing. Systemic disease: |
| | | | | | | Splenomegaly with prior splenectomy due |
| | | | | | | to a rupture |
| 41-1-09 | NPC1: Homozygous c.3122A > G | Juvenile | 9 | years | Epilepsy | Severe stance and gait ataxia and |
| | (p.Tyr1041Cys) | | | | | spasticity, dysphagia, medium |
| | | | | | | perpendicular ataxia, severe treatment |
| | | | | | | refractory epilepsy, reduced speech (but |
| | | | | | | no full anarthria), medium cognitive |
| | | | | | | decline. Systemic disease: Mild |
| | | | | | | splenomegaly. |
| 41-1-10 | NPC1: Homozygous p. Val1165Met | Adolescent/ | 49 | years | Ataxia | Severe ataxic gait with postural |
| | | Adult | | | | instability, falling backward. Moderate to |
| | | | | | | severe extremity dystonia, |
| | | | | | | disdiadochokinesis, VSGP, mild |
| | | | | | | depression/apathy, mild cognitive decline. |
| 41-1-11 | NPC1: Homozygous | Adolescent/ | 15 | years | Ataxia | Discrete stance and gait ataxia, mild |
| | c.2662C > T(p.P888S) | Adult | | | | extremity ataxia and dystonia, |
| | | | | | | disdiadochokinesia, dysarthrophonia, |
| | | | | | | VSSP. Depression and obsessive |
| | | | | | | compulsive disease. |
| 41-1-12 | NPC1: Homozygous | Adolescent/ | 15 | years | Ataxia | Moderate stance and gait ataxia, mild |
| | c.2662C > T(p.P888S) | Adult | | | | extremity ataxia and dystonia, |
| | | | | | | dysarthrophonia, VSSP. Depression and |
| | | | | | | obsessive compulsive disease. |
| 41-1-13 | NPC1: c.478T > C (p.C160R), | Early- | 2 | years | Cataplexy | Mild stance and gait ataxia, discrete |
| | c.665A > G (p.N222S), c.3106A > G | infantile | | | | dysmetria and dystonia, cataplexy, |
| | (p.T1036A) | | | | | vertical supranuclear saccade palsy, mild |
| | | | | | | epilepsy, ADHD. |
| 42-1-01 | c.2861C > T p.(Ser954Leu), Exon 19 | Juvenile | 11 | years | vertical gaze palsy | hyperkinesias, ataxia, tremor, spasticity, |
| | c.3027del p.(Lys1010Serfs*30) Exon | | | | | vertical gaze palsy |
| | 20 | | | | | |
| 42-1-02 | c.808 delG (+) c.2861 C > T | Juvenile | 17 | years | epilepsy | spasticity, tremor, ataxia, vertical gaze |
| | | | | | | palsy, epilepsy, dysphagia, insomnia |
| 43-1-01 | NPC1: c.[2861C > T]; [3182T > C] | Adolescent/ | 32 | years | Dyscoordination | Vertical supranuclear gaze palsy, |
| | | Adult | | | | dysarthria, dysphagia, tremor, bilateral |
| | | | | | | perceptive hypacusis, ataxia. |
| 43-1-02 | NPC1: c.[2096T > A]; [2861C > T] | Adolescent/ | 17 | years | Dyspraxia | Vertical supranuclear gaze palsy, |
| | | Adult | | | | dysarthria, dysphagia, pseudobulbar |
| | | | | | | syndrome, tremor, dysmetria, ataxia, |
| | | | | | | dystonic-dyskinetic movements. |
| 43-1-03 | NPC1: c.[ 2780 C > T]; c.[ 2780 C > T] | Juvenile | 19 | years | VSPG | Vertical supranuclear gaze palsy, |
| | | | | | | dysarthria, dysphagia, tremor, dysmetria, |
| | | | | | | mild bilateral perceptive hypacusis, |
| | | | | | | ataxia, dystonic-dyskinetic movements, |
| | | | | | | anxiety, depression. |
| 43-1-04 | NPC1: c. [2039dupT]; [2861C > T] | Juvenile | 8 | years | Ataxia, tremor | Vertical supranuclear gaze palsy, |
| | | | | | | dysarthria, dysphagia, tremor, dysmetria, |
| | | | | | | mild bilateral perceptive hypacusis, |
| | | | | | | ataxia, dystonic-dyskinetic movements. |
| 44-1-01 | heterozygous NPC1 c1947 + 5 G > C, | Late-infantile | 4 | years | ataxic gait, intention | vertical supranuclear gaze palsy, impaired |
| | c. 3107 C > T | | | | tremor, proximal leg | mobility- needs wheelchair when outside, |
| | | | | | weakness | ataxia, dysarthria, tremor, dysmetria, |
| | | | | | | dysphagia, cataplexy, reduced central tone |
| | | | | | | (hypotonia), cognitive delcine with |
| | | | | | | moderate learning difficulties. |
| 44-1-02 | heterozygous NPC1 c 1554-1009 | Late-infantile | 5 | years | gelastic cataplexy, | vertical supranuclear gaze palsy, intention |
| | G > A, c3182T > C p(Ile1081Thr) | | | | daytime somnolence | tremor, cataplexy, narcolepsy, bileteral |
| | | | | | | sensorineural hearing loss, ataxia, |
| | | | | | | dysarthria, dysmetria, moderate learning |
| | | | | | | difficulties, mild short term memory loss. |
| 44-3-01 | Homozygous for a previously- | Late-infantile | 4 | years | Vertical | mild ataxia, memory issues, dysmetria |
| | reported complex allele: c.1408G > C | | | | supranuclear gaze | |
| | p.(Ala470Pro) and c.2509A > G | | | | palsy | |
| | p.(Ile837Val) | | | | | |
| 44-3-02 | Compound heterozygous c.350A > G | Late-infantile | 6 | years | Ataxia, cataplexy | moderate ataxia, slurring of words, |
| | p.(Gln117Arg) and c.3182T > C | | | | | cataplexy, tremor, seizures, ocular motor |
| | p.(Ile1061Thr) | | | | | apraxia, epilepsy |
| 44-3-03 | Compound heterozygous | Juvenile | 8 | years | Ataxia, cataplexy | moderate ataxia, slurring of words, |
| | c.3182T > Cp.(Ile1061Thr) and | | | | | cataplexy |
| | c.3611_3614del | | | | | |
| | p.(Leu1204Glnfs*37) | | | | | |
| 44-3-04 | Compound heterozygous c.2713C > A | Juvenile | 6 | years | Ataxia, cataplexy | moderate ataxia, epilepsy, behavioral |
| | p.(Gln905Lys) and c.3182T > C | | | | | difficulty, severe learning disability, |
| | p.(Ile1061Thr) | | | | | vertical eye saccade eye movement defect |
| 44-4-01 | c.3493G > A (p.Val1165Met) in exon | Early- | 10 | years | Dysarthria, cognitive | Ataxia, dysarthria, cognitive impairment |
| | 23 | infantile | | | impairment (short | (short term memory impairment) |
| | | | | | term memory | |
| | | | | | impairment), ataxia | |
| | | | | | the following year | |
| 44-4-02 | p.Ser901Tyr + p.Asn222Ser; deep | Juvenile | 7 | years | Ataxia, Dyspraxia, | Ataxia, Dyspraxia, dysphagia, worsening |
| | intronic: c.1554-1009G > A | | | | Dysphagia, short | short term memory impairment and |
| | | | | | term memory | difficulty in information processing and |
| | | | | | impairment and | slow speech, epilepsy (nocturnal tonic |
| | | | | | difficulty in | clonic seizures), insomnia and night |
| | | | | | information | terrors, hydrocephalus requiring VP |
| | | | | | processing and slow | shunt. |
| | | | | | speech , Insomnia | |
| | | | | | with night terror | |
| 44-4-03 | Compound heterozygote | Juvenile | 15 | years | Ataxia, Cognitive | ataxia, tremor, Vertical Supranuclear Gaze |
| | for:c.3182T.C p.(IIe1061Thr) and | | | | impairment: | palsy, Cognitive Impairment, swallowing |
| | c.3019C.G, p.(Pro1007Ala). | | | | (problems with | difficulty, sleep disturbance |
| | | | | | memory and | |
| | | | | | concentration) - | |
| | | | | | Progressive deficits | |
| | | | | | of delayed recall; | |
| | | | | | withperseveration/ | |
| | | | | | impairment of | |
| | | | | | executive function. | |
| | | | | | Stable registration of | |
| | | | | | information and | |
| | | | | | recognition memory | |
| | | | | | Swallowing problem | |
| 44-4-04 | Compound heterozygote | Juvenile | 14 | years | Supraventricular | High frequency hearing loss (Bilateral |
| | for:c.3182T.C p.(IIe1061Thr) and | | | | gaze palsy, | hearing aids), Supraventricular gaze |
| | c.3019C.G, p.(Pro1007Ala). | | | | Tremor, Hepato- | palsy, Cognitive impairment, Tremor, |
| | | | | | splenomegaly, Mild | Hepato-splenomegaly, Anxiety |
| | | | | | Cognitive | |
| | | | | | impairment | |
| 49-3-01 | NPC1: c.2660C > T (p.Pro887Leu) | Juvenile | 15 | years | Dysarthria | Dysatrhia, bradykinesia, Spastic-ataxic |
| | and c.3019C > G (p.Pro1007Ala) | | | | | gait with rare falls, dystonic posturing, |
| | | | | | | mild dysmetria, dysarthrophonia, facial |
| | | | | | | dystonia, vertical supranuclear saccade |
| | | | | | | and gaze palsy (VSSP and VSGP), |
| | | | | | | dysarthria, mild horizontal saccadic palsy, |
| | | | | | | tremor, fine motor disturbance, mild |
| | | | | | | cognitive impairment, deficits especially |
| | | | | | | in attention and word fluency |
| | | | | | | domains. Systemic disease: |
| | | | | | | Hepatosplenomegaly |
| 49-4-01 | c.3394G > C p.A1132P homozygous | Late-infantile | 6 | years | Ataxia | Severe ataxia, dysarthria, epilepsy, |
| | | | | | | dysmetria, dysdiadochokinesia, |
| | | | | | | dysphagia, vertical ophthalmoplegia, |
| | | | | | | cognitive plateau, memory loss, mild |
| | | | | | | depression, urine incontinence |
| 49-4-02 | chr18: 21115645 C > G | Late-infantile | 4 | years | Ataxia | vertical ophthalmoplegia, cataplexia, |
| | p. Glu 1089Glnheterozygous AND | | | | | severe ataxia, dysphagia, dysmetria, |
| | chr. 18: 21118528 G > C p.Pro1007Ala | | | | | dysdiadochokinesia, aggressivity, |
| | heterozygous | | | | | incontinence, spasticity, minimal |
| | | | | | | cognitive function, memory loss, unable |
| | | | | | | to follow commands, epilepsy |
| 49-4-03 | c.3019C > G p.P1007A heterozygous | Late-infantile | 4 | years | Ataxia | mild ataxia, abnormal chewing, |
| | AND c.3104C > T p.A1035V | | | | | dysdiadochokinesia, hyperkinetic |
| | heterozygous | | | | | disorder, incontinence, minimal cognitive |
| | | | | | | function, memory loss, mild vertical |
| | | | | | | ophthalmoplegia, non-verbal |
| 49-4-04 | c.2762A > G p.Q921P heterozygous | Late-infantile | 6 | years | Cataplexy | vertical ophthalmoplegia, cataplexy, |
| | AND c.3106A > G p.T1036A | | | | | severe ataxia, dysphagia, dysmetria, |
| | heterozygous | | | | | dysdiadochokinesia, depression, |
| | | | | | | incontinence, spasticity, minimal |
| | | | | | | cognitive function, memory loss, unable |
| | | | | | | to follow commands, epilepsy |
| 49-4-05 | c.3182T.C p.I1061T heterozygous | Juvenile | 9 | years | Ataxia | Severe ataxia, dysarthria, epilepsy, |
| | and c.3104C > T p.A1035V | | | | | cataplexia, dysmetria, |
| | heterozygous | | | | | dysdiadochokinesia, dysphagia, vertical |
| | | | | | | ophthalmoplegia, cognitive plateau, |
| | | | | | | memory loss, spasticity |
| 49-4-06 | c.3182T.C p.I1061T heterozygous | Juvenile | 8 | years | Ataxia | Ataxia, dysarthria, epilepsy, dysmetria, |
| | and c.3104C > T p.A1035V | | | | | dysarthria, dysdiadochokinesia, |
| | heterozygous | | | | | dysphagia, mild vertical ophthalmoplegia, |
| | | | | | | cognitive plateau, memory loss |
| 49-4-07 | c.2728G > A pGly910Ser homozygous | Adolescent/ | 21 | years | Dystonia | ataxia, severe dysphagia, dysmetria, |
| | | Adult | | | | dysdiadochokinesia, massive dystonia, |
| | | | | | | cognitive plateau, memory loss, mild |
| | | | | | | vertical ophthalmoplegia |
| 49-4-08 | c.1211G > A p.R404Q Heterozygous | Juvenile | 7 | years | Ataxia | vertical ophthalmoplegia, narcolepsy, |
| | AND c.3493G > A p.V1165M | | | | | severe ataxia, dysphagia, dysmetria, |
| | heterozygous | | | | | dysdiadochokinesia, incontinence, |
| | | | | | | spasticity, cognitive dysfunction |
| | | | | | | (plateau), memory loss, epilepsy |
| 49-4-09 | c.1114C > T p.R372W heterozygous | Adolescent/ | 29 | years | Ataxia | Ataxia, delayed speech acquisitions, |
| | AND c.3322dupG p.A1108Gfs*13 | Adult | | | | tremor, dysdiadochokinesia, dysphagia, |
| | heterozygous | | | | | vertical ophthalmoplegia, dystonia |
| 49-5-01 | NPC1 [c.3182T > C, (p.I1061T)]; | Early- | 1 | year | muscular | ataxia, vertical gaze palsy, delayed motor |
| | [c.2893C > T, (p.Q965*)] | infantile | | | hypotonia, delayed | development, impaired expressive |
| | | | | | motor milestones | language |
| 49-5-02 | NPC1 [c.2849T > C, | Late-infantile | 3 | years | impaired/delayed | ataxia, dysarthria, vertical gaze palsy, |
| | (p. V950G)]; [c.3182T > C, (p.I1061T)] | | | | language | seizures, hearing impairment |
| | | | | | development | |
| 49-5-03 | NPC1 c.3019C > G, | Juvenile | 10 | years | ataxia | ataxia, dysarthria, vertical gaze palsy, |
| | (p.P1007A)homozygous | | | | | cataplex, reduced alterness |
| 49-5-04 | NPC1 c.3019C > G, | Adolescent/ | 20 | years | ataxia, vertical gaze | ataxia, dysarthria, vertical gaze palsy |
| | (p.P1007A)homozygous | Adult | | | palsy | |
| 49-5-06 | NPC1 [c.3019C > G, (p.P1007A)]; | Juvenile | 9 | years | ataxia, tremor | ataxia, dysarthria, vertical gaze plasy, |
| | [c.2090T > C, (p.V697A)] | | | | | tremor, ADHD |
| 49-5-07 | NPC1 [c.3246-25A > G]; [c.1926G > C, | Juvenile | 9 | years | ataxia | ataxia, dysarthria, vertical gaze palsy, |
| | (p.M641I)] | | | | | schizophrenia, sleep disturbance |
| 49-5-08 | NPC1 [c.3246-25A > G]; [c.1926G > C, | Juvenile | 10 | years | ataxia, vertical gaze | ataxia, dysarthria, vertical gaze palsy, |
| | (p.M641I)] | | | | palsy | sleep disturbance |
| 61-1-01 | p.R615C heterozygote and p.G1015V | Juvenile | 11 | years | Seizuresexecutive | Mute and limited comprehension to |
| | heterozygote | | | | and memory | simple verbal instruction. Assist for |
| | | | | | impairment | essential activities of daily life and unable |
| | | | | | | to do instrumental activities of daily life. |
| | | | | | | Independent gait with severe ataxia and |
| | | | | | | spasticity. Able to uphold posture. |
| | | | | | | Intermittent impulsive and incoherent |
| | | | | | | motor actions. Significant vertical and |
| | | | | | | horizontal gaze palsy. Occasional |
| | | | | | | moderate dysphagia. Insomnia. |
| 61-1-02 | c.3019C > G (p.Pro1007Ala) | Juvenile | 14 | years | Clumsy fine motor | Good verbal and non-verbal |
| | heterozygous and c.3182T > C | | | | and learning deficits | communication. Able to complete |
| | (p.lle1061Thr) heterozygous | | | | | essential activities of daily life and |
| | | | | | | minimal support for instrumental |
| | | | | | | activities of daily life. Vertical gaze palsy |
| | | | | | | on examination. Mild bradykinesia and |
| | | | | | | subtle fine motor impairment (intention |
| | | | | | | tremor and coordination). |
| 61-1-03 | c.1654 + 6T > A homozygote | Adolescent/ | 25 | years | Ataxic gait, | Comprehend simple verbal instruction |
| | | Adult | | | Executive | and conversation. Assist for essential |
| | | | | | impairment | activities of daily life and unable to do |
| | | | | | | instrumental activities of daily life. |
| | | | | | | Scanning speech with brisk speech |
| | | | | | | intonation. Intermittent |
| | | | | | | hypersomnolence.Independent gait with |
| | | | | | | severe ataxia and spasticity. Able to |
| | | | | | | uphold posture. Significant vertical and |
| | | | | | | horizontal gaze palsy. Occasional |
| | | | | | | moderate dysphagia. |
| 61-1-04 | c.3019 C > G (p.Pro1007Ala) | Late-infantile | 10 | years | Ataxia, dysarthria | Wheelchair or 1 person full-assist for |
| | heterozygous and c.3182T > C | | | | | mobility. Comprehend simple verbal |
| | (p.lle1061Thr) heterozygous | | | | | instruction and conversation. Full assist |
| | | | | | | for essential ADL and unable to do |
| | | | | | | instrumental activities of daily life. |
| | | | | | | Scanning speech with brisk speech |
| | | | | | | intonation. Able to uphold posture <30 |
| | | | | | | s. Significant vertical and horizontal |
| | | | | | | gaze palsy. Moderate dysphagia, required |
| | | | | | | thickened fluid. |
| 61-1-05 | p.Arg518Trp heterozygote and | Adolescent/ | 21 | years | Executive | Good verbal and non-verbal |
| | p.Asp1156Ser heterozygote | Adult | | | impairment | communication. Able to complete |
| | | | | | | essential activities of daily life and |
| | | | | | | minimal support for instrumental |
| | | | | | | activities of daily life. Part- time |
| | | | | | | supervised manual labor |
| | | | | | | employment. Independent mobility |
| | | | | | | without aid. Vertical gaze palsy on |
| | | | | | | examination. Mild bradykinesia and |
| | | | | | | subtle fine motor impairment (intention |
| | | | | | | tremor and coordination). Subtle |
| | | | | | | dysarthria. |
| 61-1-06 | p.Asp1097Asn heterozygote and | Adolescent/ | 52 | years | Ataxic gait, | Good verbal and non-verbal |
| | pTyr1088Cys heterozygote | Adult | | | Executive | communication. Able to complete |
| | | | | | impairment | essential ADL and minimal support for |
| | | | | | | instrumental activities of daily |
| | | | | | | life.Independent mobility with walking |
| | | | | | | frame or walking stick. Mild bradykinesia |
| | | | | | | and ataxic gait. Vertical gaze palsy on |
| | | | | | | examination. Mild bradykinesia and |
| | | | | | | subtle fine motor impairment (intention |
| | | | | | | tremor and coordination). Subtle |
| | | | | | | dysarthria. Anxiety disorder. |

*Disease start was defined as the earliest of 2 dates: start date of the Medical History item or the date of genetic confirmation of the disease. Disease duration was calculated as the difference in days between the V then converted to months. Partially missing dates were imputed using the first day of the month and the first month of the year in case month and/or day were missing.
**Phenotype was coded based on date of disease start and per the following categories: Early-infantile: <2 years; Late-infantile: 2-<6 years; Juvenile: 6-<15 years; Adolescent/Adult: >=15 years.
***As reported by the patient's Investigator

TABLE 7

| | | | | | | | |
|---|---|---|---|---|---|---|---|
| | | NALL | | | Placebo | | |
| | | MEAN | | | Mean | | |
| | NALL | Change | | Placebo | Change | NALL vs | |
| | EOT | from | | EOT | from | Placebo | |
| EOT NALL | n= | Baseline | EOT Placebo | n= | Baseline | difference¹ | 95% CI |

Primary Endpoint²

| | | | | | | | | |
|---|---|---|---|---|---|---|---|---|
| Scale for the Assessment and | 13.71 | 59 | −1.97 | 15.21 | 58 | −0.60 | −1.28 | −1.91 |
| Rating of Ataxia (SARA) ³ | (SD = 7.68) | | (SD = 2.43) | (SD = 7.27) | | (SD = 2.39) | | to −0.65 |
| Modified Scale for the Assessment | 11.37 | 59 | −1.66 | 12.47 | 58 | −0.67 | −0.96 | −1.45 |
| and Rating of Ataxia (mSARA) | (SD = 5.81) | | (SD = 1.97) | (SD = 5.34) | | (SD = 1.74) | | to −0.46 |

Secondary Endpoints⁴

| | | | | | | | | |
|---|---|---|---|---|---|---|---|---|
| Investigator's Clinical Global | 3.3 | 30 | −0.7 | 3.9 | 30 | −0.1 | −0.6 | −1.071 |
| Impression of Improvement⁵ | (SD = 0.9) | | | (SD = 0.9) | | | | to −0.13 |
| Caregiver Clinical Global | 3.6 | 27 | −0.4 | 4.3 | 25 | 0.3 | −0.7 | −1.17 |
| Impression of Improvement⁵ | (SD = 0.9) | | | (SD = 0.9) | | | | to 0.23 |
| Patient Clinical Global Impression | 3.3 | 24 | −0.7 | 3.8 | 26 | −0.2 | −0.5 | 1.1 0.1 |
| of Improvement⁵ | (SD = 1.0) | | | (SD = 1.1) | | | | |
| Spinocerebellar Ataxia Functional | −0.17 | 57 | 0.05 | −0.27 | 56 | −0.02 | 0.07 | 0 |
| Index (SCAFI) | (S D = 0.98) | | (SD = 0.27) | (SD−0.99) | | (SD = 0.31) | | to 0.14 |
| Modified Disability Rating Scale | 0.447 | 59 | −0.030 | 0.478 | 58 | −0.001 | −0.029 | −0.048 |
| (mDRS)⁶ | (SD = 0.157) | | (SD-0.060) | (SD = 0.136) | | (SD0.061) | | to −0.01 |

Exploratory Endpoint³

| | | | | | | | | |
|---|---|---|---|---|---|---|---|---|
| Niemann-Pick Disease type C | 17.6 | 58 | −0.3 | 18.2 | 58 | 0.1 | −0.51 | −1.2 |
| Clinical Severity Scale | (SD = 6.8) | | (SD = 2.2) | (SD = 6.9) | | (SD = 2. I) | | to 0.18 |

¹For the SARA/mSARA the LS Mean Difference is given
²Last Observation Carried Forward (LOCF) approach was applied to patient 49-4-07 Visit 4 and 43-1-03 Visit 6 for SARA/mSARA
³ SARA primary endpoint two-sided p < 0.001
⁴Only reported data was included in the CGI/exploratory endpoint analyses
⁵CGI-I was calculated for Period 1 using a paired t-test. The change from baseline is the reported change from a score of “4, no change”.
⁶mDRS was scaled from 24 total points to a nought-to-one (0 to 1)
EOT = End of Treatment

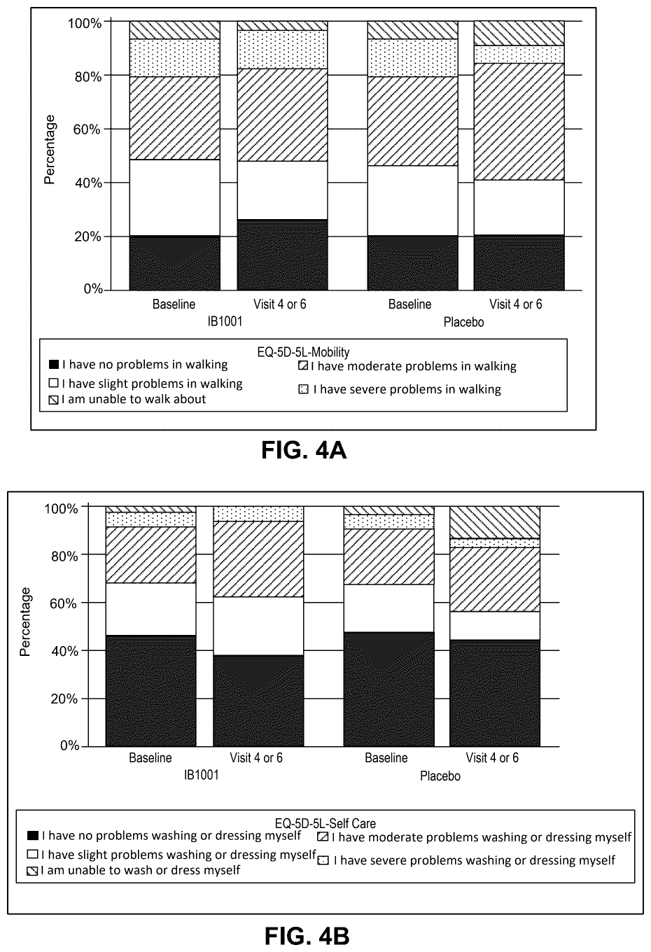

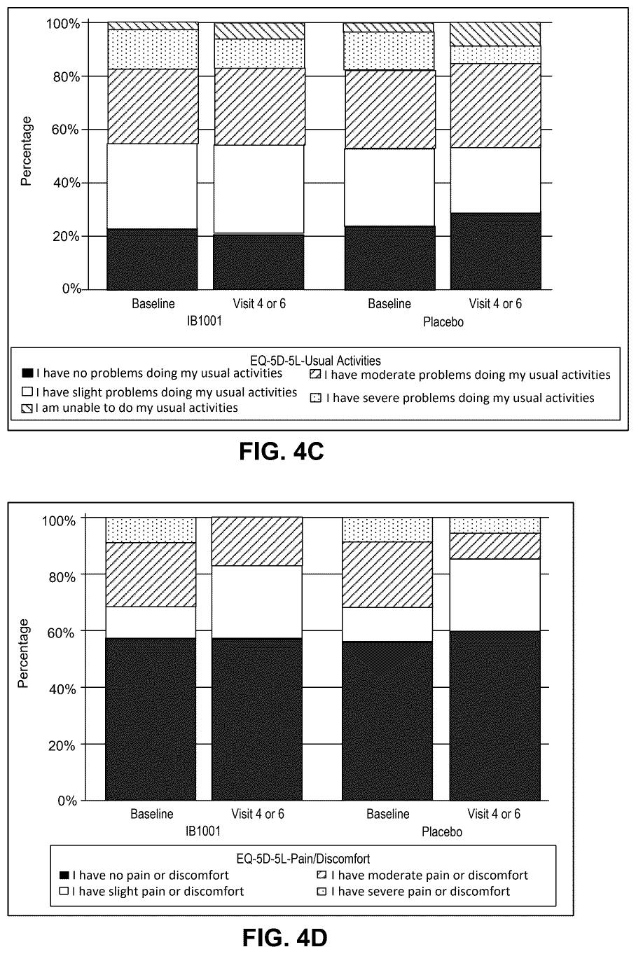

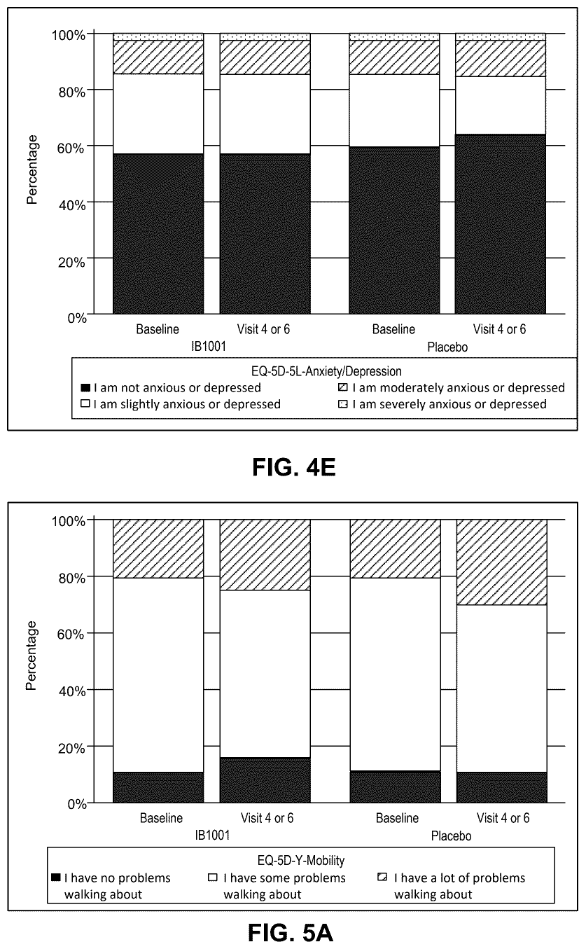

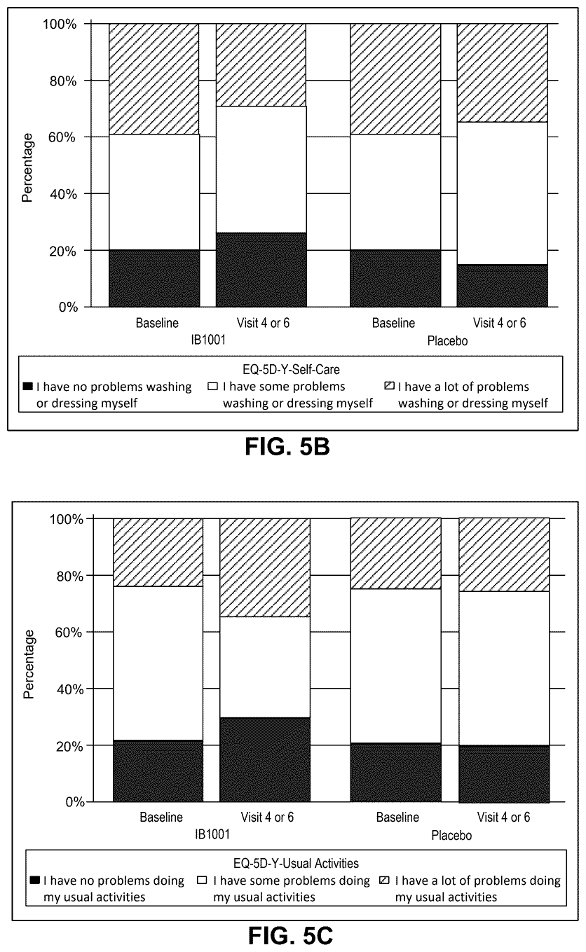

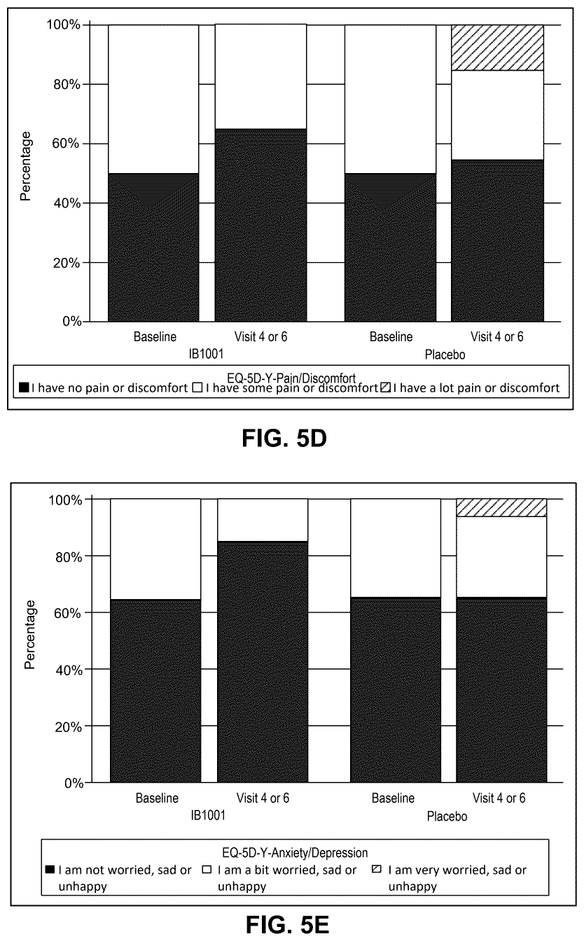

Quality of life improvements with NALL versus placebo were captured by the EuroQol (EQ)-5D-5L assessment, which demonstrated a decrease in patients' reported rates of moderate to severe problems with mobility and an increase in patients' reported ability to perform self-care. In comparison, there were increased reports of severe pain or discomfort on placebo compared to NALL. See FIGS. 4A-E. For youth (EQ-5D-Y; used for patients aged <18 years), there were reported improvements in mobility, self-care, ability to perform usual activities (e.g. wash and dress independently), and decreases in pain/discomfort and anxiety/depression. See FIGS. 5A-E.

### Example 2

Efficacy was measured using a modified version of the Scale for Assessment and Rating of Ataxia (SARA), referred to as the functional SARA (fSARA), according to the human clinical study described in EXAMPLE 1. See AQNEURSA™ PI at https://www.accessdata.fda.gov/drugsatfda_docs/label/2024/219132s000lb1.pdf. The SARA is a clinical assessment tool that assesses gait, stability, speech, and upper and lower limb coordination across 8 individual domains. The fSARA consists only of gait, sitting, stance, and speech disturbance domains of the original SARA with modifications to the scoring responses. Each domain was rescored from 0 to 4, where 0 is the best neurological status and 4 the worst, with a total score ranging from 0 to 16.

The fSARA score was assessed at baseline, 6 weeks, 12 weeks (the end of Period I), 18 weeks, and 24 weeks (the end of Period II). The estimated mean fSARA total score was 5.1 when patients were treated with NALL and 5.6 when patients were treated with placebo. The estimated treatment difference for the fSARA total score was −0.4 (95% CI:−0.7,−0.2) (Table 9).

TABLE 9

| | fSARA Total Score | |
|---|---|---|
| | Treatment Sequence 1: | Treatment Sequence 2: |
| Variable | AQNEURSA - Placebo | Placebo - AQNEURSA |
| Baseline | N = 30 | N = 30 |
| Mean (SD) | 5.2 (3.0) | 6.3 (3.3) |
| Period I | N = 29 | N = 30 |
| Mean (SD) | 4.5 (2.6) | 6.0 (3.4) |
| Period II | N = 28* | N = 30 |
| Mean (SD) | 5.1 (2.8) | 5.6 (3.1) |

Estimated Mean fSARA Score (SE) by Treatment

| | |
|---|---|
| AQNEURSA | 5.1 (0.1) |
| Placebo | 5.6 (0.1) |
| Treatment | −0.4 (−0.7, −0.2)** |
| Difference (95% CI) | |

CI = confidence interval; SD = standard deviation; SE = standard error.
*Two patients did not have an assessment at the end of Period II (week 24).
**Two-sided p-value < 0.001

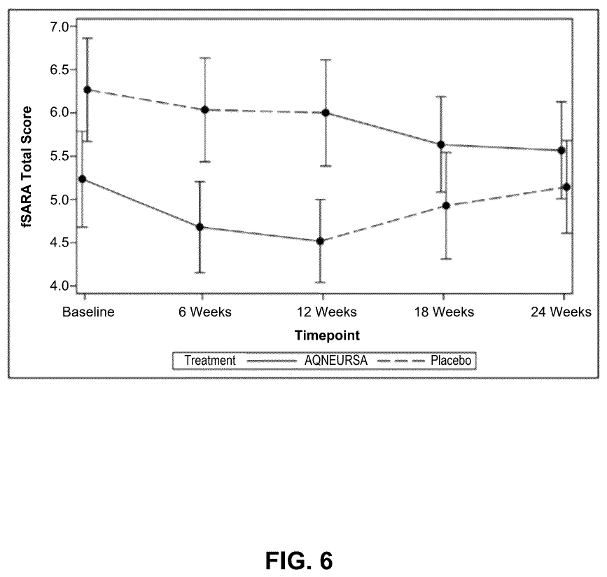

Patients who received NALL in Period I followed by placebo in Period II (Treatment Sequence 1) showed a greater improvement in the fSARA score in Period I with a mean change from baseline of −0.5 (SD 1.2), compared to Period II with a mean change from baseline of 0 (1.5). Similarly, patients who received placebo in Period I followed by NALL in Period II (Treatment Sequence 2) experienced greater improvement in the fSARA score while receiving NALL in Period II with a mean change of −0.7 (0.9), compared to a mean change of −0.3 (0.9) in Period I (FIG. 6).

### Example 3

Extension Phase Study with NALL

Patients who completed the final scheduled visit of the clinical trial described in EXAMPLE 1 (“Parent Study” or “IB1001-301”) were eligible to continue into an open-label Extension Phase (EP) under the same trial protocol. The EP was conducted at the same trial sites, and with the same Investigators, as the Parent Study. Eligible participants were those who (a) completed the Parent Study Visit 6, (b) for whom the Investigator determined continued treatment with NALL may be in their best interest, and (c) who (or their legal representative) provided written informed consent to continue in the EP.

### Extension Phase Study Design

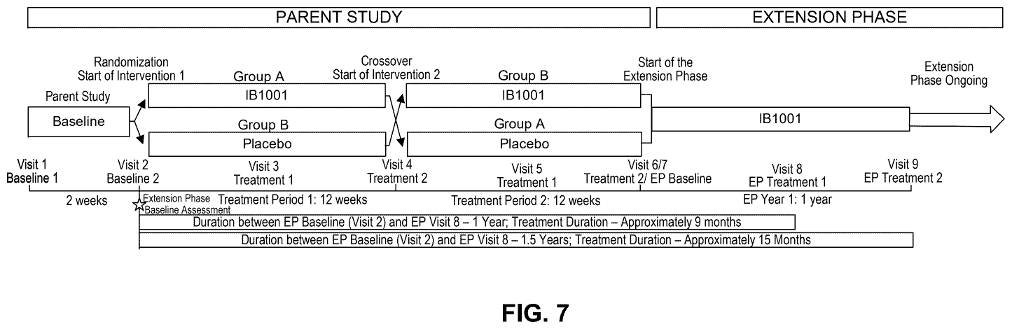

The EP is an open-label study. The trial consisted of a baseline visit, conducted in tandem with the last visit of the Parent Study (Visit 6). The EP visit was called “Visit 7” (4 patients had independent Visit 7 visits conducted 28, 42, 57, and 64 days after Visit 6). Following this baseline visit, patients received open-label treatment with NALL for a minimum of 1 year (365+/−14 days). Visits occurred at 6 months (Visit 8, 180+/−14 days) and after 1 year (Visit 9, 365+/−14 days) (FIG. 7). The EP is ongoing. A prespecified analysis was planned after all patients who participated in the Parent Study and continued into the EP had completed Visit 9.

Given the placebo-controlled crossover design of the Parent Study, n=27 (51%) of patients were receiving NALL at EP Baseline Visit 7. Accordingly, it was pre-specified that the Parent Study Randomization Baseline Visit (Visit 2) was utilized as the “baseline” visit for the EP analysis. The approximate duration between Baseline (Visit 2) and Visit 8 was 12 months and 18 months between Baseline (Visit 2) and Visit 9. Over these durations, patients received treatment with NALL for approximately 9 months and 15 months, respectively (FIG. 7).

Patients aged ≥13 years or aged 4-12 years weighing ≥35 kg received 4 g/day of orally administered NALL (granules in a sachet for suspension in 40 mL water, orange juice, or almond milk) three times per day (2 g in the morning, 1 g in the afternoon, and 1 g in the evening). Patients aged 4-12 years weighing <35 kg received weight-tiered doses two or three times per day based on an approximate total dose of 0.1 g/kg/day.

### Outcomes

The primary endpoint of the Extension Phase was the modified 5-domain Niemann-Pick disease type C Clinical Severity Scale (5-domain NPC-CSS), a five-item (Ambulation, Cognition, Fine motor skills, Speech, and Swallow) clinical rating scale ranging from 0-25, where 0 is the best neurological status and 25 the worst 4. Each domain is rated on a scale of 0-5. The 5-domain NPC-CSS is an abbreviated assessment tool derived from the 17-domain NPC-CSS developed specifically by Yanjanin et al., Am J Med Genet B Neuropsychiatr Genet; 2010; 153B. Accessed at: pubmed.ncbi.nlm.nih.gov/19415691 as a clinical outcome assessment to characterize and quantify disease progression in patients with NPC. The 17-domain NPC-CSS consists of 9 major domains (Ambulation, Cognition, Eye movement, Fine motor skills, Memory, Seizures, Speech, Swallow, Hearing) and 8 modifiers (Auditory Brainstem Response, Behavior, Gelastic cataplexy, Hyperreflexia, Incontinence, Narcolepsy, Psychiatric and Respiratory). In this study, the widely used 15-domain NPC-CSS was utilized (which excludes the hearing and auditory brainstem response domains) Fields et al., Trials; 2023;24. Accessed at: pubmed.ncbi.nlm.nih.gov/37248494. The 15-domain NPC-CSS has a total score for overall neurological status ranging from 0 (best) to 54 (worst) and was utilized as an exploratory endpoint.

The definitions of response with respect to 5- and 15-domain NPC-CSS scores were selected to measure deviations from the expected trajectory of disease progression established in the published natural history studies in patients with NPC. Yanjanin et al. first reported, based on a cross-sectional evaluation of 37 NPC patients, disease progression could be modelled on the 17-domain NPC-CSS by the following equation: St0+x=St0+1.87x; where St0 is the initial score and St0+x is the predicted future score after x years. Mengel et al., Orphanet J Rare Dis; 2020; 15. Accessed at: pubmed.ncbi.nlm.nih.gov/3322879 subsequently reported in a prospective observational study of 36 NPC patients, a mean (±SD) increase of 1.4±2.9 on the 5-domain NPC-CSS (corresponding to an annualized progression rate of 1.5 points) and 2.7±4.0 on the NPC-CSS (excluding hearing) after 12 months.

In the above studies, a linear annualized increase (independent of age of disease onset, and similar in all patients) has been documented on the 5-domain/NPC-CSS scales, reflecting the progressive neurodegenerative nature of NPC. Therefore, a higher score indicates a clinical worsening (disease progression), while a lower 5-domain/NPC-CSS score indicates a clinical improvement and disease modification. A 0-point change represents a stabilization of disease progression, which is also a significant clinical benefit for rapidly progressive, neurodegenerative diseases such as NPC.

In a 12-month, double-blind, randomized placebo-controlled trial with arimoclomol, the mean progression after 12 months in the 16 patients receiving placebo was 2.15 points on the 5-domain NPC-CSS and 2.7 points on the NPC-CSS. Mengel et al., Orphanet J Rare Dis; 2020; 15. Accessed at: pubmed.ncbi.nlm.nih.gov/3322879. However, for the sake of this analysis, the conservative, validated linear natural history cohort values have been utilized as the basis for comparison, potentially underestimating the clinical deterioration in untreated patients.

Exploratory endpoints included the Scale for the Assessment and Rating of Ataxia (SARA), an eight-item (gait, stance, sitting, and speech disturbance, as well as the finger-chase test, the nose-to-finger test, the fast-alternating-hand-movements test, and the heel-along-shin slide test) clinical rating scale that incorporates functional assessments of gait, balance, speech, fine motor function, and upper/lower extremity function; scores range from 0 to 40, with lower scores indicating better neurological status Schmitz-Hübsch et al., 2006; 66:1717-1720. The 4-domain NPC-CSS (the 5-domain NPC-CSS with rescored swallow domain and excluding the cognition domain), which served as the basis for the FDA marketing authorization of the combination therapy Arimoclomol and Miglustat, was also analyzed. MIPLYFFA-Prescribing-Information.pdf. 2024. Accessed at: zevra.com/documents/MIPLYFFA-Prescribing-Information.pdf.

Safety assessments included monitoring for adverse events (whereby the site investigators or their delegates assessed the relation of the event to NALL), clinical laboratory testing and full pharmacokinetic sampling, physical examination, evaluation of vital signs, and electrocardiogra

### Statistical Analysis

The number of patients entering the Extension Phase was determined by the number of patients completing the parent study (Visit 6) and who consented to participate in the open-label follow-up with NALL. The primary endpoint was defined as the numerical difference of the 5-domain NPC-CSS value for patients treated with NALL from baseline (Visit 2) versus 12 months (Visit 8) and 18 months (Visit 9) evaluated against benchmark annual mean rates of progression from the historical cohorts under the standard of care of 1.5 points annually for the 5-domain and 1.87 points annually for the 15-domain NPC-CSS.

An independent-sample t-test at a two-sided 5% significance level was used to test the null hypotheses that the mean change from the Extension Baseline on the 5-domain NPC-CSS and the 15-domain NPS-CSS score is equal to or greater than the 12 months or 18 months change expected in the natural history cohorts. The mean and standard deviation for the 18-month historical control cohort was modelled based on the formulas for the annualized increase. Point estimates and 95% confidence intervals of the mean difference are presented. The mean differences are also presented for the primary 5-domain NPC-CSS for key subgroups, including pediatric and adult patients and patients on miglustat or not receiving background miglustat.

The mean change from the Extension Baseline and 95% confidence intervals are computed for the exploratory endpoints: 4-domain NPC-CSS and SARA. A one-sample t-test at two-sided 5% significance level was used to test whether the change in SARA from Extension Baseline differed from zero after 12 and 18 months no historical data exists on the 4-domain NPC-CSS, this endpoint was reported descriptively.

For the 4-, 5-, 15-domain NPC-CSS and the SARA score, descriptive tables are presented with data available from all published or publicly presented previous natural history cohorts and clinical trial cohorts. The data is presented for each scale at 12 months (consistent with what is available in the literature and public domain) (see Tables 10-13). The mean and SD (if available) for each cohort are presented, along with the mean difference from the NALL EP cohort. If available, for clinical trial cohorts treated with drug therapies, the data with and without miglustat on each scale at 12 months is presented.

The primary analysis was performed according to the modified intention-to-treat (mITT) principle, used to estimate the treatment effect regardless of discontinuation and to provide a perspective of the treatment effect across the entire population. The Extension Phase Modified Intention to Treat analysis set (mITTe) consisted of all patients aged 4 years and older who receive at least one dose of study drug (N-Acetyl-L-Leucine) in the Extension Phase, and with NPC-CSS scores at Extension Analysis Baseline (Visit 2)) and during the Extension Phase Treatment Period I (Visit 8 or Visit 9).

The Safety Analysis Extension Phase Set (SAFe) consisted of all patients who received at least one dose of study drug in the Extension Phase. The safety, integrity, and feasibility of the trial were monitored by an independent data safety monitoring board (DSMB) consisting of three independent, non-participating members (including two clinicians and a statistician).

TABLE 10
12 Months at Baseline

| | | | | Mean |
|---|---|---|---|---|
| | | Mean | | Difference vs |
| | | Change | % Change | IB1001-301 |
| | | from | from | EP Cohort |
| Cohort | n= | Baseline* | Baseline** | (NALL) |
| IB1001-301 Extension Phase Cohort | 49 | −0.27 | −18% | N/A |
| (NALL)*** | | (SD = 2.42) | | |
| Patients on NALL + Miglustat | 41 | −0.17 | −11% | N/A |
| | | (SD = 2.53) | | N/A |
| Patients on NALL (no miglustat) | 8 | −0.75 | −50% | |
| | | (SD = 1.83) | | |
| Mengel et al. 2020⁶ (Natural History | 32 | +1.5 | +100% | +1.77 |
| Cohort) | | (SD−3.16) | | |
| Arimoclomol Treated Cohort (from | 34 | +0.76 | +51% | +1.03 |
| Arimoclomol Phase II/III trial)^{T}**** | | (SD = 2.4) | | |
| Patients on Arimoclomol + Miglustat⁷ | 26 | −0.06 | −4% | +0.11 |
| | | (N/A) | | |
| Patients on Arimoclomol - (no miglustat)⁷ | 8 | +4.2 | +280% | +4.95 |
| | | (N/A) | | |
| Placebo Treated Cohort (from | 16 | +2.15 | +143% | +2.42 |
| Arimoclomol Phase II/III trial)⁷***** | | (SD = 2.25) | | |

*A higher score represents disease worsening; a lower score reflects disease improvement
**Calculated based on an annualized progression rate of 1.5 points (representing a +100% rate of annual progression); a positive value reflects disease progression; 0 reflects no change; a negative value reflects disease reversal.
***Includes patients treated with NALL (no Miglustat) (n = 8) and patients treated with NALL + Miglustat (n = 41)
****Includes patients treated with Arimoclomol (without miglustat) (n = 8) and patients treated with Arimoclomol + miglustat combination therapy (n = 26)
*****Includes patients treated with Placebo (no Miglustat) (n = 4) and Placebo + Miglustat (n = 12)

TABLE 11
12 Months at Baseline

| | | | | Mean |
|---|---|---|---|---|
| | | Mean | | Difference vs |
| | | Change | % Change | IB1001-301 |
| | | from | from | EP Cohort |
| Cohort | n= | Baseline* | Baseline** | (NALL) |
| IB1001-301 Extension Phase Cohort | 49 | −0.00 | −3% | N/A |
| (NALL)*** | | (SD = 3.25) | | |
| Yanjamin et al. 2010⁵ (Natural History | 37 | +1.87 | +100% | +1.87 |
| Cohort) | | (SD = 1.1) | | |
| Ory et al. 2017¹⁹ (Natural History | 21 | +2.92 | +156% | +2.92 |
| Cohort) | | (SD = 1.24) | | |
| Mengel et al. 2020⁶ (Natural History | 32 | +2.94 | +157% | +2.94 |
| Cohort) | | (SD = 4.5) | | |
| Arimoclomol Treated Cohort**** | 34 | +1.20 | +64% | +1.20 |
| | | (SD = 4.75) | | |
| Placebo Treated Cohort (from | 16 | +2.81 | +150% | +2.81 |
| Arimoclomol Phase II/III trial)***** | | (SD = 4.2) | | |

*A higher score represents disease worsening; a lower score reflects disease improvement
**Calculated based on an annualized progression rate of 1.87 points (representing a +100% rate of annual progression); a positive value reflects disease progression; 0 reflects no change; a negative value reflects disease reversal.
***Includes patients treated with NALL (no Miglustat) (n = 8) and patients treated with NALL + Miglustat (n = 41)
****Includes patients treated with Arimoclomol (without miglustat) (n = 8) and patients treated with Arimoclomol + miglustat combination therapy (n = 26)
*****Includes patients treated with Placebo (no Miglustat) (n = 4) and Placebo + Miglustat (n = 12)

TABLE 12
12 Months from Baseline

| | | | | Mean Difference |
|---|---|---|---|---|
| | | Mean Change | | vs IB1001-301 EP |
| Cohort | n= | from Baseline* | | Cohort (NALL) |
| IB1001-301 Extension Phase Cohort | 49 | −0.59 | (SD = 1.79) | N/A |
| (NALL)** | | | | |
| Patients on NALL + Miglustat | 41 | −0.59 | (SD = 1.86) | N/A |
| Patients on NALL (no miglustat) | 8 | −0.63 | (SD = 1.51) | N/A |
| Arimoclomol Treated Cohort (from | 34 | +0.62 | (SD = 0.39) | +1.21 |
| Arimoclomol Phase II/III trial)²⁰*** | | | | |
| Patients on Arimoclomol + Miglustato | 22 | −0.2 | (SD = 1) | +0.39 |
| Patients on Arimoclomol - (no miglustat)²⁰ | 8 | +4.13 | (N/A) | +4.27 |
| Placebo Treated Cohort (from | 16 | +2.12 | (SD = 0.59) | +2.71 |
| Arimoclomol Phase II/III trial)²⁶**** | | | | |
| Patients on Placebo + Miglustat⁹ | 12 | +2.0 | (SD = 0.7) | +2.59 |
| Patients on Placebo (no miglustat)²⁰ | 3 | +1.98 | (N/A) | +2.57 |

*A higher score represents disease worsening; a lower score reflects disease improvement
**Includes patients treated with NALL (no Miglustat) (n = 8) and patients treated with NALL + Miglustat (n = 41)
***Includes patients treated with Arimoclomol (no miglustat) (n = 8) and patients treated with Arimoclomol + miglustat combination therapy (n = 26)
****Includes patients treated with Placebo (no Miglustat) (n = 4) and Placebo + Miglustat (n = 12)

TABLE 13
12 Months from Baseline

| | | | Mean |
|---|---|---|---|
| | | Mean | Difference vs |
| | | Change | IB1001-301 |
| | | from | EP Cohort |
| Cohort | n= | Baseline* | (NALL) |
| IB1001-301 Extension Phase Cohort | 51 | −1.88 | N/A |
| (NALL)** | | (SD = 2.89) | |
| Arimoclomol Treated Cohort (from | 34 | +1.06 | +2.94 (std |
| Arimoclomol Phase II/III trial)⁷*** | | (SD = 3.66) | err = 0.75) |
| Placebo Treated Cohort (from | 16 | +0.78 | +2.66 (std |
| Arimoclomol Phase II/III trial)⁷**** | | (SD = 1.62) | err = 0.57) |

*A higher score represents a worsening of neurological status; a lower score represents an improvement in neurological status
**Includes patients treated with NALL (no Miglustat) (n = 8) and patients treated with NALL + Miglustat (n = 43)
***Includes patients treated with Arimoclomol (no miglustat) (n = 8) and patients treated with Arimoclomol + miglustat combination therapy (n = 26)
****Includes patients treated with Placebo (no Miglustat) (n = 4) and Placebo + Miglustat (n = 12)

### Results

### Study Population

Fifty three patients were enrolled in the Extension Phase (aged 5 to 67 years). The demographic and baseline clinical characteristics of the enrolled patients are presented in Table 14. Forty-nine patients qualified for the primary Modified Intention-to-Treat Extension Phase (mITTe) analysis set (92.4%), which included all patients dosed who had an NPC-CSS score at baseline and at least one Extension Phase treatment visit (Visit 8 or Visit 9). Three patients did not have a NPC-CSS score at baseline due to accidentally missed assessment, and thus were not included; one patient was withdrawn after Visit 7 following withdrawal of consent.

TABLE 14

| Parameter | Statistic/Category | Total (n = 53) | |
|---|---|---|---|
| Age | Pediatric (<18 years) | 22 | (41.5%) |
| | Adult (>=18 years) | 31 | (58.5%) |
| Gender (n (%)) | Female | 24 | (45.3%) |
| | Male | 29 | (54.7%) |
| Race (n (%)) | American Indian or Alaska native | 0 | (0.0%) |
| | Asian | 2 | (3.8%) |
| | Black or African American | 0 | (0.0%) |
| | Native Hawaiian or other Pacific Islander | 0 | (0.0%) |
| | White | 47 | (88.7%) |
| | Other | 4 | (7.5%) |
| Age at diagnosis group (n (%)) | Early-infantile (<2 years) | 9 | (17.0%) |
| | Late-infantile (2 to <6 years) | 13 | (24.5%) |
| | Juvenile (6 to <15 years) | 21 | (39.6%) |
| | Adolescent/Adult (>=15 years) | 10 | (18.9%) |
| Dose group (n (%)) | Age 4-12 years - 15 to <25 kg -2 g per day | 5 | (9.4%) |
| | Age 4-12 years - 25 to <35 kg - 3 g per day | 3 | (5.7%) |
| | Age 4-12 years - >=35 kg or age >=13 | 45 | (84.9%) |
| | years - 4 g per day | | |
| Miglustat at baseline* (n (%)) | Yes | 45 | (84.9%) |
| | No | 8 | (15.1%) |

*Indicates concurrent miglustat use throughout the duration of the trial

Visit 8 occurred approximately 1 year after the Baseline Visit (Visit 2) during which patients received treatment with NALL for approximately 9 months (mean duration of 268 [min 233, max 287] days out of mean 354 days [min 317, max 371]). Visit 9 occurred approximately 18 months after the Baseline Visit (Visit 2) during which patients received treatment with NALL for approximately 15 months (mean duration of 453 [min 435, max 556] days out of 539 days [min 520, max 633]) (FIG. 7). The EP remains ongoing for additional years of long-term follow up.

### Efficacy

### Primary Endpoint

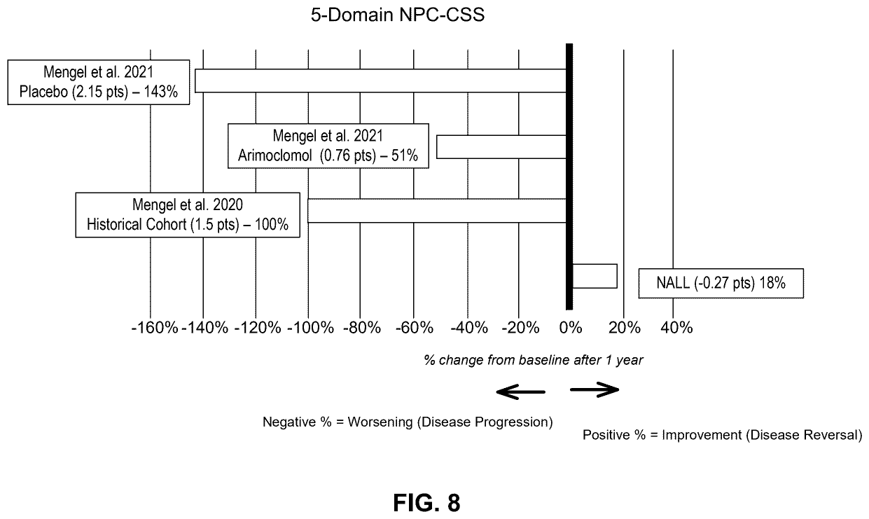

The mean (±SD) baseline (Visit 2) on the 5-domain NPC-CSS was 11.10 (±4.73). After 12 months (Visit 8), the mean change from baseline was −0.27±2.42 points with NALL versus 1.5±3.1 points in the historical cohort [mean difference −1.77 points; 95% confidence interval [CI], −3.05 to −0.48; p=0.009]. After 18 months (Visit 9), the mean change from baseline was +0.045±2.95 with NALL versus 2.25 (4.74) in the historical cohort [mean difference −2.20 points; 95% confidence interval [CI], −4.06 to −0.35; p=0.023). Table 10/FIG. 8 reports and depicts the change in the 5-domain NPC-CSS with NALL compared with all published historical cohorts as well as clinical trial cohorts.

Subgroup analysis of pediatric versus adult patients demonstrated consistent benefit with NALL. After 12 months (Visit 8), the mean change from baseline was −0.10±3.16 points for pediatric patients and −0.39±1.73 for adult patients. After 18 months (Visit 9), the mean change from baseline was −0.21±3.49 for pediatric patients and +0.24±2.52 for adult patients.

### Exploratory Endpoints

The mean (±SD) baseline (Visit 2) score on the 15-domain NPC-CSS was 18.28 (±6.98). After 12 months (Visit 8), the mean change from baseline was 0.0±3.25 points with NALL versus 1.87±1.09 points in the historical cohort [mean difference −1.87; 95% confidence interval [CI], −2.83 to −0.87; p<0.001]. After 18 months (Visit 9), the mean change from baseline was 0.43±4.65 with NALL versus 2.81±1.64 in the historical cohort [mean difference −2.37; 95% confidence interval [CI], −3.85 to −0.90; p=0.002]. Table 11 reports the change on the 15-domain NPC-CSS with NALL compared with all published historical cohorts as well as published clinical trial cohorts.

The mean (±SD) baseline (Visit 2) score on the 4-domain NPC-CSS was 8.14 (±3.58). After 12 months (Visit 8), the mean change from baseline was −0.59±1.79 points with NALL. After 18 months (Visit 9), the mean change from baseline was −0.27±2.29 NALL. Table 12 reports the change on the 4-domain NPC-CSS with NALL compared with all publicly available cohorts.

The mean (±SD) baseline (Visit 2) score on the SARA score was 15.91 (7.65). After 12 months (Visit 8), the mean change from baseline was −1.88±2.89 points with NALL (95% confidence interval [CI], −2.70 to −1.07; p (mean change=0)<0.001). After 18 months (Visit 9), the mean change from baseline was −1.64±3.24 with NALL (95% confidence interval [CI], −2.59 to −0.69, (mean change=0, p<0.001). There was no significant difference between these values and those from the completion of the Parent Study; rather, the improvements on the SARA were sustained across long-term treatment. Table 13 reports the change on the SARA with NALL compared with all published cohorts.

### Safety

No Treatment-Emergent Adverse Events (TEAEs) led to premature discontinuation of the trial. No TEAEs occurred in more than 10% of patients on NALL and no patients had TEAEs that were assessed as by the investigator as related to NALL.

No trial drug-related serious adverse events (SAEs) or deaths occurred. Results of plasma and urine tests, vital signs, and ECG recordings were normal or rated as clinically non-significant. Adherence to trial drug was high as shown by treatment compliance and the regular urine analyses for prohibited medication.

### Classification of Evidence

This study provides Class IV evidence that N-acetyl-L-leucine reduces disease progression in Niemann-Pick Type C.

### DISCUSSION

The major findings of this study are as follows: in adult and pediatric patients with NPC, treatment with NALL was associated with a 118% reduction in annual disease progression after one year compared to a natural history control cohort, on the 5-domain NPC-CSS primary endpoint, reflecting a significant improvement in the patient's condition from 1-year prior. After 1 year, there was a reduction of −0.27 points from baseline (compared to a 1.5-point increase on the historical cohort) on a 25-point scale used to assess neurological status in multiple domains and disease progression in NPC, and a change of 0.0 after 1.5 years (versus the expected +2.25 point change). This improvement is to date the most significant of any agent formally investigated in NPC (Table 10, 11, 12; FIG. 8).

In the randomized, double-blind Parent Study, NALL demonstrated a significant improvement in neurological signs and symptoms after 12 weeks. The improvement in neurological manifestations and status demonstrated with NALL versus placebo on the SARA scale were maintained after 12 and 18 months of treatment, demonstrating a statistically significant and clinically meaningful improvement 10 and the long-term effects of NALL. No new safety signals and no drug-related serious adverse events were observed during the follow-up, reinforcing the benign safety profile of the molecule.

The improvement on the 5-domain NPC-CSS scale with NALL is clinically meaningful according to the validation of the 5-domain NPC-CSS, which demonstrated that a 1-point worsening on the 5-domain NPC-CSS constitutes a clinically meaningful change for caregivers/parents and physicians (e.g., a 1-point change or greater represents a clinically meaningful transition reflecting loss of complex function and increased disability), and therefore, preventing a 2-point worsening would be a viable treatment goal 11. Treatment with NALL not only prevented worsening (e.g. halted disease progression) on the 5-domain NPC-CSS, but led to an improvement, demonstrating a neuroprotective and disease-modifying benefit. This benefit was demonstrated both in patients who were and were not receiving Miglustat, reflecting NALL's efficacy as a stand-alone and consistent with results from the placebo-controlled parent study (where NALL improved neurological signs, symptoms, and quality of life irrespective of background Miglustat use). Bremova-Ertl et al., N Engl J Med; 2024;390. Accessed at: pubmed.ncbi.nlm.nih.gov/38294974/. (Table 10).

Limitations of the trial include the open-label design. Analyses were not adjusted for multiple comparisons. The data of the historical cohort were not made publicly available, thus it was not possible to match the historical cohort with the IB1001-301 EP patient population. Nevertheless, the IB1001-301 EP study population was significantly larger (31 vs. 54 patients with continued follow-up). However, due to the inclusion criteria of the Parent Study, the EP did not include patients aged <4 years, asymptomatic patients, or patients with advanced disease who would not be able, or reliably able, to complete functional assessments. Although the comparison to historical cohorts was valuable, the lack of a placebo control group in the extension phase introduced a potential for bias. Finally, in the context of this progressive neurodegenerative disorder, longer periods of observation will be beneficial to determine the full safety and efficacy data of NALL.

In conclusion, this study provides additional evidence of the benefits of NALL treatment. NALL acts as a metabolic modulator, rebalancing dysregulated energy metabolism and is pleiotropic in its effects. NALL has demonstrated both rapid improvements in neurological manifestations, as well as a long-term, disease-modifying, neuroprotective effect for patients with NPC.

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents and publications cited herein are fully incorporated by reference in their entirety.

1. A method of treating or preventing Niemann-Pick disease type C (NPC), or a neurological symptom thereof, in a subject in need thereof, the method comprising administering a therapeutically effective amount of N-acetyl-DL-leucine, or a pharmaceutically acceptable salt thereof, or N-acetyl-L-leucine, or a pharmaceutically acceptable salt thereof, to the subject, wherein the subject has any one or more of the following NPC1 gene mutations:
c.3019C>G p. (Pro1007Ala);
c.1654+5G>A, p. (?);
c.275A>G (p.Gln92Arg);
c.1918G>A (p.Gly640Arg);
c.[1918G>A](p. [Gly640Arg](±) [3451G>A] p.Ala1151Thr);
c.2861C>T, p. (Ser954Leu);
c.1574A>T p. (asp525Val);
c.306T>G p.Tyr102*;
c.180G>T p.Gln60His;
homozygous c.1918G>A (p.Gly640Arg);
c.3451G>A (p.Ala1151Thr);
c.2474 A>G p. (Tyr825Cys);
c.2780C>T (p.A1a927Val);
c.1295delG (p.Gly432Aspfs*17);
homozygous T3128->C (Ile1061->Thr);
p.11061T;
p.D948N;
homozygous c.3246-5_3246-7del;
c.2819C<T (p.Ser940Leu);
c.2657G>T;
c.2932C>T;
c.2692G>A;
c.3773delCC;
homozygous c.3122A>G (p.Tyr1041Cys);
homozygous p. Val1165Met;
homozygous c.2662C>T (p.P888S);
c.478T>C (p.C160R);
c.665A>G (p.N222S);
c.3106A>G (p.T1036A);
c.2861C>T p. (Ser954Leu) in exon 19;
c.3027del p. (Lys1010Serfs*30) in exon 20;
c.[2096T>A];
c.[2861C>T];
c.[2780 C>T];
c.[2780 C>T];
c.[2039dupT];
heterozygous c1947+5 G>C, c.3107 C>T;
heterozygous c1554-1009 G>A, c3182T>C p (Ile1081Thr);
homozygous c.1408G>C p. (Ala470Pro);
homozygous c.2509A>G p. (Ile837Val);
compound heterozygous c.3182T>C p. (Ile1061Thr);
compound heterozygous c.3611_3614del p. (Leu1204GInfs*37);
compound heterozygous c.2713C>A p. (Gln905Lys);
compound heterozygous c.3182T>C p. (Ile1061Thr);
c.3493G>A (p. Val1165Met) in exon 23;
p.Ser901Tyr+p.Asn222Ser;
c.1554-1009G>A;
compound heterozygous c.3182T.C p. (IIe1061Thr);
compound heterozygous c.3019C.G p. (Pro1007Ala);
c.2660C>T (p.Pro887Leu);
c.3019C>G (p.Pro1007Ala);
homozygous c.3394G>C p.A1132P;
heterozygous chr18:21115645 C>G p.Glu1089Gln;
heterozygous chr. 18:21118528 G>C p.Pro1007Ala;
heterozygous c.3019C>G p.P1007A;
heterozygous c.3104C>T p.A1035V;
heterozygous c.2762A>G p.Q921P;
heterozygous c.3106A>G p.T1036A;
heterozygous c.3182T.C p.I1061T;
heterozygous c.3104C>T p.A1035V;
homozygous c.2728G>A pGly910Ser;
heterozygous c.1211G>A p.R404Q;
heterozygous c.3493G>A p.V1165M;
heterozygous c.1114C>T p.R372W;
heterozygous c.3322dupG p.A1108Gfs*13;
[c.3182T>C, (p.I1061T)];
[c.2893C>T, (p.Q965*)];
[c.2849T>C, (p.V950G)];
homozygous c.3019C>G, (p.P1007A);
[c.2090T>C, (p.V697A)];
[c.3246-25A>G];
[c.1926G>C, (p.M641I)];
heterozygous p.R615C;
heterozygous p.G1015V;
homozygous c.1654+6T>A;
heterozygous p.Arg518Trp;
heterozygous p.Asp1156Ser;
heterozygous p.Asp1097Asn; and/or
heterozygous p.Tyr1088Cys.

2. The method of claim 1, wherein a first neurological symptom of NPC in the subject is any one or more of the following neurological symptoms:
cognitive impairment;
cognitive decline;
cerebellar ataxia;
clumsiness;
attention-deficit/hyperactivity disorder (ADHD);
dystonia;
supranuclear gaze palsy;
ataxia;
dysarthrophonia;
epilepsy;
cataplexy;
vertical gaze palsy;
dyspraxia;
tremor;
gelastic cataplexy;
vertical supranuclear gaze palsy;
daytime somnolence;
dysarthria;
dysphagia;
insomnia;
supraventricular gaze palsy;
hepato-splenomegaly;
muscular hypotonia;
executive impairment; and/or
memory impairment.

3. The method of claim 1, wherein the subject is less than two years old.

4-6. (canceled)

7. The method of claim 1, wherein administering N-acetyl-DL-leucine, or N-acetyl-L-leucine to the subject results in an improvement over baseline in at least one clinical assessment of neurological signs and symptoms of NPC, wherein the at least one clinical assessment is the Scale for the Assessment and Rating of Ataxia (SARA), the modified SARA (mSARA), the functional SARA (fSARA), the modified Disability Rating Scale (mDRS), Niemann-Pick type C Clinical Severity Scale (NPC-CSS), 5-Domain NPC-CSS, and/or the Spinocerebellar Ataxia Functional Index (SCAFI).

8-9. (canceled)

10. The method of claim 7, wherein the SARA comprises a change from baseline in the SARA total score of at least −0.6.

11-18. (canceled)

19. The method of claim 7, wherein the fSARA comprises a change from baseline in the fSARA total score of at least −0.2.

20-27. (canceled)

28. The method of claim 1, wherein N-acetyl-DL-leucine or N-acetyl-L-leucine is administered to the subject for at least 1 week.

29-31. (canceled)

32. The method of claim 1, wherein administering N-acetyl-DL-leucine or N-acetyl-L-leucine to the subject results in an improvement over baseline in at least one quality of life (QOL) assessment.

33. The method of claim 32, wherein the at least one QOL assessment is the Clinical Global Impression of Severity and Improvement Scale (CGI-I), the EuroQol (EQ) 5Q-5D-5L/Y questionnaire, and/or the Visual Analogue Scale (VAS).

34-36. (canceled)

37. The method of claim 1, wherein a total dose of about 0.7 g to about 8 g of N-acetyl-DL-leucine or N-acetyl-L-leucine is administered to the subject per day.

38. (canceled)

39. The method of claim 3, wherein the N-acetyl-DL-leucine or N-acetyl-L-leucine is administered once a day, twice a day, three times, or four times a day to the subject to achieve the total daily dose.

40. The method of claim 34, wherein the N-acetyl-DL-leucine or N-acetyl-L-leucine is administered to the subject as a tablet or as an oral suspension.

41. The method of claim 1, where the subject has one or more homozygous NPC1 gene mutations or one or more heterozygous NPC1 gene mutations.

42. (canceled)

43. The method of claim 1, wherein the subject has one, two, three, or four NPC1 gene mutations.

44-46. (canceled)

47. The method of claim 1, wherein the N-acetyl-DL-leucine or N-acetyl-L-leucine is administered to the subject in combination with one or more additional therapeutic agents, wherein the one or more additional therapeutic agents comprise miglustat, levetiracetam, valproate, sertraline, donezepil, clonazepam, risperidone, melatonin, ramipril, metronidazole, lamotrigine, piracetam, 2-hydroxypropyl-β-cyclodextrin (HPBCD), vorinostat, lovastatin, rapamycin, and/or arimoclomol.

48-49. (canceled)

50. A method of treating a subject having NPC, or a neurological symptom thereof, the method comprising:
(a) determining whether any one or more of the following NPC1 gene mutations are present or absent in a biological sample taken from the subject:
c.3019C>G p. (Pro1007Ala);
c.1654+5G>A, p. (?);
c.275A>G (p.Gln92Arg);
c.1918G>A (p.Gly640Arg);
c.[1918G>A](p. [Gly640Arg](±) [3451G>A] p.Ala1151Thr);
c.2861C>T, p. (Ser954Leu);
c.1574A>T p. (asp525Val);
c.306T>G p.Tyr102*;
c.180G>T p.Gln60His;
homozygous c.1918G>A (p.Gly640Arg);
c.3451G>A (p.Ala1151Thr);
c.2474 A>G p. (Tyr825Cys);
c.2780C>T (p.Ala927Val);
c.1295delG (p.Gly432Aspfs*17);
homozygous T3128->C (Ile1061->Thr);
p.I1061T;
p.D948N;
homozygous c.3246-5_3246-7del;
c.2819C<T (p.Ser940Leu);
c.2657G>T;
c.2932C>T;
c.2692G>A;
c.3773delCC;
homozygous c.3122A>G (p.Tyr1041Cys);
homozygous p. Val1165Met;
homozygous c.2662C>T (p.P888S);
c.478T>C (p.C160R);
c.665A>G (p.N222S);
c.3106A>G (p.T1036A);
c.2861C>T p. (Ser954Leu) in exon 19;
c.3027del p. (Lys1010Serfs*30) in exon 20;
c.[2096T>A];
c.[2861C>T];
c.[2780 C>T];
c.[2780 C>T];
c.[2039dupT];
heterozygous c1947+5 G>C, c.3107 C>T;
heterozygous c1554-1009 G>A, c3182T>C p (Ile1081Thr);
homozygous c.1408G>C p. (Ala470Pro);
homozygous c.2509A>G p. (Ile837Val);
compound heterozygous c.3182T>C p. (Ile1061Thr);
compound heterozygous c.3611_3614del p. (Leu1204GInfs*37);
compound heterozygous c.2713C>A p. (Gln905Lys);
compound heterozygous c.3182T>C p. (Ile1061Thr);
c.3493G>A (p. Val1165Met) in exon 23;
p.Ser901Tyr+p.Asn222Ser;
c.1554-1009G>A;
compound heterozygous c.3182T.C p. (Ile1061Thr);
compound heterozygous c.3019C.G p. (Pro1007Ala);
c.2660C>T (p.Pro887Leu);
c.3019C>G (p.Pro1007Ala);
homozygous c.3394G>C p.A1132P;
heterozygous chr18:21115645 C>G p.Glu1089Gln;
heterozygous chr. 18:21118528 G>C p.Pro1007Ala;
heterozygous c.3019C>G p.P1007A;
heterozygous c.3104C>T p.A1035V;
heterozygous c.2762A>G p.Q921P;
heterozygous c.3106A>G p.T1036A;
heterozygous c.3182T.C p.I1061T;
heterozygous c.3104C>T p.A1035V;
homozygous c.2728G>A pGly910Ser;
heterozygous c.1211G>A p.R404Q;
heterozygous c.3493G>A p.V1165M;
heterozygous c.1114C>T p.R372W;
heterozygous c.3322dupG p.A1108Gfs*13;
[c.3182T>C, (p.I1061T)];
[c.2893C>T, (p.Q965*)];
[c.2849T>C, (p.V950G)];
homozygous c.3019C>G, (p.P1007A);
[c.2090T>C, (p.V697A)];
[c.3246-25A>G];
[c.1926G>C, (p.M641I)];
heterozygous p.R615C;
heterozygous p.G1015V;
homozygous c.1654+6T>A;
heterozygous p.Arg518Trp;
heterozygous p.Asp1156Ser;
heterozygous p.Asp1097Asn; and/or
heterozygous p. Tyr1088Cys; and
(b) administering a therapeutically effective amount of N-acetyl-DL-leucine, or a pharmaceutically acceptable salt thereof, or N-acetyl-L-leucine, or a pharmaceutically acceptable salt thereof, to the subject if any one or more of the NPC1 gene mutations are present in the biological sample.

51. A method, comprising administering a therapeutically effective amount of N-acetyl-DL-leucine, or a pharmaceutically acceptable salt thereof, or N-acetyl-L-leucine, or a pharmaceutically acceptable salt thereof, to a subject in need thereof, wherein:
(a) the subject has NPC, or a neurological symptom thereof; and
(b) any one or more of the following NPC1 gene mutations is present in a biological sample taken from the subject:
c.3019C>G p. (Pro1007Ala);
c.1654+5G>A, p. (?);
c.275A>G (p.Gln92Arg);
c.1918G>A (p.Gly640Arg);
c.[1918G>A](p. [Gly640Arg](±) [3451G>A] p.Ala1151Thr);
c.2861C>T, p. (Ser954Leu);
c.1574A>T p. (asp525Val);
c.306T>G p.Tyr102*;
c.180G>T p.Gln60His;
homozygous c.1918G>A (p.Gly640Arg);
c.3451G>A (p.Ala1151Thr);
c.2474 A>G p. (Tyr825Cys);
c.2780C>T (p.Ala927Val);
c.1295delG (p.Gly432Aspfs*17);
homozygous T3128->C (Ile1061->Thr);
p.I1061T;
p.D948N;
homozygous c.3246-5_3246-7del;
c.2819C<T (p.Ser940Leu);
c.2657G>T;
c.2932C>T;
c.2692G>A;
c.3773delCC;
homozygous c.3122A>G (p.Tyr1041Cys);
homozygous p. Val1165Met;
homozygous c.2662C>T (p.P888S);
c.478T>C (p.C160R);
c.665A>G (p.N222S);
c.3106A>G (p.T1036A);
c.2861C>T p. (Ser954Leu) in exon 19;
c.3027del p. (Lys1010Serfs*30) in exon 20;
c.[2096T>A];
c.[2861C>T];
c.[2780 C>T];
c.[2780 C>T];
c.[2039dupT];
heterozygous c1947+5 G>C, c.3107 C>T;
heterozygous c1554-1009 G>A, c3182T>C p (Ile1081Thr);
homozygous c.1408G>C p. (Ala470Pro);
homozygous c.2509A>G p. (Ile837Val);
compound heterozygous c.3182T>C p. (Ile1061Thr);
compound heterozygous c.3611_3614del p. (Leu1204GInfs*37);
compound heterozygous c.2713C>A p. (Gln905Lys);
compound heterozygous c.3182T>C p. (Ile1061Thr);
c.3493G>A (p. Val1165Met) in exon 23;
p.Ser901Tyr+p.Asn222Ser;
c.1554-1009G>A;
compound heterozygous c.3182T.C p. (IIe1061Thr);
compound heterozygous c.3019C.G p. (Pro1007Ala);
c.2660C>T (p.Pro887Leu);
c.3019C>G (p.Pro1007Ala);
homozygous c.3394G>C p.A1132P;
heterozygous chr18:21115645 C>G p.Glu1089Gln;
heterozygous chr. 18:21118528 G>C p.Pro1007Ala;
heterozygous c.3019C>G p.P1007A;
heterozygous c.3104C>T p.A1035V;
heterozygous c.2762A>G p.Q921P;
heterozygous c.3106A>G p.T1036A;
heterozygous c.3182T.C p.I1061T;
heterozygous c.3104C>T p.A1035V;
homozygous c.2728G>A pGly910Ser;
heterozygous c.1211G>A p.R404Q;
heterozygous c.3493G>A p.V1165M;
heterozygous c.1114C>T p.R372W;
heterozygous c.3322dupG p.A1108Gfs*13;
[c.3182T>C, (p.I1061T)];
[c.2893C>T, (p.Q965*)];
[c.2849T>C, (p.V950G)];
homozygous c.3019C>G, (p.P1007A);
[c.2090T>C, (p.V697A)];
[c.3246-25A>G];
[c.1926G>C, (p.M641I)];
heterozygous p.R615C;
heterozygous p.G1015V;
homozygous c.1654+6T>A;
heterozygous p.Arg518Trp;
heterozygous p.Asp1156Ser;
heterozygous p.Asp1097Asn; and/or
heterozygous p.Tyr1088Cys.

52. (canceled)

53. The method of claim 1, the method comprises administering a therapeutically effective amount of N-acetyl-L-leucine to the subject.

54. (canceled)

55. A kit for carrying out the method of claim 52, the kit comprising N-acetyl-L-leucine and instructions for administering N-acetyl-L-leucine to the subject.
